(19) United States
(12) Patent Application Publication
(10) Pub. No.: US 2024/0124924 A1
(43) Pub. Date: Apr. 18, 2024

(54) HIGH-THROUGHPUT ASSESSMENT OF EXOGENOUS POLYNUCLEOTIDE- OR POLYPEPTIDE-MEDIATED TRANSCRIPTOME PERTURBATIONS

(71) Applicants: THE BROAD INSTITUTE, INC., Cambridge, MA (US); MASSACHUSETTS INSTITUTE OF TECHNOLOGY, Cambridge, MA (US); THE GENERAL HOSPITAL CORPORATION, Boston, MA (US)

(72) Inventors: Aziz Al'Khafaji, Cambridge, MA (US); Frances Keer, Cambridge, MA (US); Paul Blainey, Cambridge, MA (US); Nir Hacohen, Boston, MA (US)

(73) Assignees: THE BROAD INSTITUTE, INC., Cambridge, MA (US); MASSACHUSETTS INSTITUTE OF TECHNOLOGY, Cambridge, MA (US); THE GENERAL HOSPITAL CORPORATION, Boston, MA (US)

(21) Appl. No.: 18/547,258

(22) PCT Filed: Feb. 22, 2022
(86) PCT No.: PCT/US2022/017294

§ 371 Date: Aug. 21, 2023

Related U.S. Application Data
(60) Provisional application No. 63/152,542, filed on Feb. 23, 2021.

(51) Int. Cl. C12Q 1/6844; C12Q 1/6869

(52) U.S. Cl. CPC C12Q 1/6844; C12Q 1/6869

(57) ABSTRACT

The present disclosure relates to methods and compositions for enhanced assessment of exogenous polynucleotide and/or polypeptide-mediated transcriptional perturbations at high throughput and single cell/droplet levels of resolution. In embodiments, nucleic acid fusions of exogenous polynucleotide(s) and associated target transcript(s) are produced within individually sequestered or discretely identifiable cells/lysates and analyzed for exogenous polynucleotide mediated perturbations across a vast population of droplets/cells within individual reactions. Kits for performance of the methods are also provided.

### CROSS-REFERENCE TO RELATED APPLICATION

The present application is related to and claims priority under 35 U.S.C. § 119(e) to U.S. provisional patent application No. 63/152,542, entitled “High-Throughput Assessment of Exogenous Polynucleotide- or Polypeptide-Mediated Transcriptome Perturbations,” filed Feb. 23, 2021. The entire content of the aforementioned patent application is incorporated herein by this reference.

### FIELD OF THE INVENTION

The invention relates generally to methods and compositions for physical and informational linking of key cellular oligonucleotides to a target set of expressed genes at the single cell level in a highly parallel fashion.

### BACKGROUND OF THE INVENTION

Experimental assays such as CRISPR screens are powerful approaches that uncover gene interaction networks which modulate cellular behavior. Traditional CRISPR screens, however, are limited in their ability to report the complex transcriptomic consequences of a particular perturbation as the primary assay read out is that of guide RNA (gRNA) enrichment. Methods such as CROP-seq have addressed this limitation by enabling the sequencing of expressed gRNAs in single-cell gene expression workflows (Datlinger et al. Nature Methods. 14: 297-301). While informative, CROP-seq is substantially hampered by its inability to efficiently scale (<10,000 cells)—a key metric for successful screens. Accordingly, a need exists for an improved method for performing CRISPR screens using large libraries of gRNAs distributed across large populations of cells (e.g., tens of thousands to millions of cells) while also identifying individual gRNA-associated transcriptional perturbations at the level of individual cells.

### BRIEF SUMMARY OF THE INVENTION

The current disclosure relates, at least in part, to the discovery of a method for obtaining perturbation-linked transcriptional data, for perturbations mediated by individually identifiable gRNAs within a cell, at a scale that allows for tens of thousands to millions of cells to be surveyed in a single experiment. In certain aspects, the instant disclosure addresses the throughput limitations confronted by previous CRISPR screening and single cell transcriptome profiling approaches such as CROP-seq, by performing an overlap extension amplification step upon cellular transcripts and cell-resident exogenous nucleic acids (e.g., gRNAs or gRNA identifiers) that splices together cellular transcript sequences and cell-resident exogenous nucleic acids (e.g., gRNAs or gRNA identifiers). In aspects, a streamlined workflow, termed “Stitch-Seq”, is provided, which functions by physically linking exogenous nucleic acids (e.g., gRNAs or other exogenous, optionally modulatory, nucleic acids, or expressed barcodes as proxies for such exogenous nucleic acids) to a target set of expressed transcripts in single cells. These linked exogenous nucleic acids/transcripts of interest are then sequenced, enabling the association of exogenous nucleic acid perturbation and gene expression levels. By taking this targeted approach to linked exogenous nucleic acid/gene expression read out and circumventing the use of a bead barcode for linkage of a particular polynucleotide's expression or abundance to expression or abundance of a set of polynucleotides, the processes of the instant disclosure possess substantially enhanced throughput (e.g., capable of achieving throughput of between ten thousand and one billion cells (1×10⁴-1×10⁹ cells)), enabling the scale necessary for robust transcriptional based CRISPR-screens. Thus, in embodiments of the instant disclosure, individual cells from a CROP-seq-type perturbation library are isolated, e.g., encapsulated in an oil emulsion, segregated into individual microwells, etc., and the expressed mRNA transcripts of interest in such cells are stitched to the cell's cognate gRNA via overlap extension RT-PCR. The native pairing of the gRNA and several transcripts of interest at the single cell level thereby allows for the coupling of targeted gene expression alterations associated with perturbation at significantly higher throughput than existing methods (e.g., Perturb-seq, CROP-seq). It is further contemplated that the approach of the instant disclosure also enables assessment of protein variants and/or protein libraries for impact upon intracellular signaling. For example, a library of transcription factors can be assessed to identify changes in expression of target genes. In such transcription factor embodiments, the transcription factors can be stitched to the target genes/transcripts, thereby associating the library of transcription factors to their respective downstream effects.

In one aspect, the instant disclosure provides a method for identifying within a population of individually sequestered or discretely identifiable cells one or more target transcripts and one or more exogenous polynucleotides in an individual cell, the method involving: (a) preparing or providing a population of individually sequestered or discretely identifiable cells, where a plurality of the cells harbor one or more exogenous polynucleotides or include a nucleic acid vector capable of expressing one or more exogenous polynucleotides and are in contact with nucleic acid amplification reagents and a plurality of oligonucleotides including: (i) a first pair of oligonucleotide primers for amplifying an exogenous polynucleotide in the individually sequestered or discretely identifiable cell; and (ii) a second pair of oligonucleotide primers for amplifying a target transcript of the individually sequestered or discretely identifiable cell, where the first pair of oligonucleotide primers possesses a primer having a 5′-terminal region of sequence that is the same or complementary to a 5′-terminal region of sequence of a primer of the second pair of oligonucleotide primers, where the 5′-terminal region that is the same or complementary between the first pair of oligonucleotide primers and the second pair of oligonucleotide primers is of sufficient length to allow for amplification-mediated joining of an amplicon of the first pair of oligonucleotide primers and an amplicon of the second pair of oligonucleotide primers into a fused amplicon, where the individually sequestered or discretely identifiable cell is lysed to render contents of the cell accessible to enzymes and/or oligonucleotides (e.g., nucleic acid amplification reagents and/or paired oligonucleotide primers) in a manner that maintains the sequestering or discrete identification of the lysed cell contents; (b) performing polymerase-mediated primer extension and optionally thermal cycling (e.g., for performance of RT-PCR, though isothermal amplification approaches are expressly contemplated herein, thus thermal cycling is optional in certain embodiments) upon the population of lysed cell contents under conditions suitable for generating fused amplicons including the amplicon of the first pair of oligonucleotide primers and the amplicon of the second pair of oligonucleotide primers by overlap extension, thereby generating fused amplicons within the individually sequestered or discretely identifiable lysed cell contents; (c) recovering fused amplicons from the population of lysed cell contents; and (d) obtaining sequence information from the fused amplicons using a sequencing method capable of obtaining sequences from both ends of individual fused amplicon sequences and identifying as a pair the sequences obtained from both ends of the same individual fused amplicon, thereby identifying in the population of individually sequestered or discretely identifiable cells one or more target transcripts and one or more exogenous polynucleotides within the individually sequestered or discretely identifiable cell.

In certain embodiments, the individually sequestered or discretely identifiable cells: are droplet-encapsulated or emulsion-encapsulated; are present in a hydrogel (optionally where the population of individually sequestered or discretely identifiable cells has been split and pool labeled); are present in a microfluidic chip; or are present in an array. Optionally, the population of individually sequestered or discretely identifiable cells is present in a microwell array and/or a plate. Optionally, the microwell array is a microwell array having a sub-nanoliter fluid volume per well (e.g., 900 microwells per array, 3600 microwells per array, 12,300 microwells per array, 14,400 microwells per array, 24,000 microwells per array, 41,600 microwells per array, 80,000 microwells per array, etc.) and/or the plate is a 96-well or 384-well plate.

In certain additional embodiments, cells used in the instant disclosure can be fixed prior to dropletization. Exemplary fixatives for use in the instant disclosure include, without limitation, methanol and paraformaldehyde (PFA), among others known in the art.

In some embodiments, a single-stranded or double-stranded nucleic acid (e.g., a ssDNA, ssRNA, or dsDNA) is also spiked, at known concentration, into a droplet-based (or otherwise sequestered) Stitch PCR of the instant disclosure, which enables calculation of relative expression of natively captured genes. For example, a known sequence of a ssDNA, ssRNA, or dsDNA at a known concentration is spiked into the PCR mix prior to dropletization. The standard (known sequence) is then able to stitch to a gRNA, allowing for normalization of each cell's natively captured gene counts to the spiked single-stranded nucleic acid standard.

In one embodiment, the nucleic acid amplification reagents include one or more of the following reagents: Polymerase Chain Reaction (PCR) reagents, Recombinase Polymerase Amplification (RPA) reagents, Rolling Circle Amplification (RCA) reagents, and/or Loop-mediated isothermal amplification (LAMP) reagents or other isothermal amplification reagents. Optionally, the nucleic acid amplification reagents include or are PCR reagents. Optionally, the nucleic acid amplification reagents include or are reverse transcriptase PCR (RT-PCR) reagents.

In certain embodiments, the polymerase-mediated primer extension and optionally thermal cycling performed upon the population of lysed cell contents under conditions suitable for generating fused amplicons comprising the amplicon of the first pair of oligonucleotide primers and the amplicon of the second pair of oligonucleotide primers by overlap extension includes performing one or more rounds of amplification via Polymerase Chain Reaction (PCR), Recombinase Polymerase Amplification (RPA), Rolling Circle Amplification (RCA), and/or Loop-mediated isothermal amplification (LAMP) or other isothermal amplification, upon the population of lysed cell contents. Optionally, PCR and thermal cycling are performed upon the population of lysed cell contents. Optionally, reverse transcriptase PCR (RT-PCR) and thermal cycling are performed upon the population of lysed cell contents.

While the instant disclosure specifically exemplifies processes that involve identifying an exogenous polynucleotide (i.e. sgRNA as particularly exemplified herein) that is part of a nucleoprotein complex, the methods of the instant disclosure are expressly contemplated as applicable to a wide range of exogenous polynucleotides (including, e.g., a wide range of expressed exogenous polynucleotides), meaning that it is expressly contemplated to employ, e.g., open reading frames (ORFs) such as RNA pol I, RNA pol II, and RNA pol III products, lineage barcodes, or exogenously added nucleic acid conjugates such as CITE-seq, hash-tags, and lipid-modified oligos as examples of exogenous nucleic acids and/or in place of transcripts in the current methods. It is also expressly contemplated that sgRNAs can be employed as lineage barcodes with or without a CRISPR effector protein. Additionally, extant polynucleotide designs or expression constructs can be modified to include particular 5′ and/or 3′ ends to facilitate amplification and overlap extension linkage to target polynucleotide products. In one example, Streptococcus pyogenes sgRNAs are modified to additionally contain a fixed 5′ adapter end, facilitating amplification and overlap extension linkage to a set of target polynucleotide products. Currently standard Streptococcus pyogenes sgRNAs do not have fixed 5′ ends thus necessitating specialized constructs such as CROP-seq to capture this sgRNA information. Because CROP-seq vectors have a number of limitations, expanded utility is thereby achieved.

In certain embodiments, the population of individually sequestered or discretely identifiable cells harbors or expresses a polynucleotide-guided protein capable of interacting with the one or more exogenous polynucleotides.

In embodiments, the one or more exogenous polynucleotides is capable of interacting with a polynucleotide-guided protein.

In some embodiments, the one or more exogenous polynucleotides include a nucleic acid sequence that identifies expression of one or more exogenous polynucleotides capable of interacting with a polynucleotide-guided protein.

In certain embodiments, identifying in the population of individually sequestered or discretely identifiable cells one or more target transcripts and one or more exogenous polynucleotides also identifies the one or more target transcripts and the one or more exogenous polynucleotides as co-expressed.

In some embodiments, the population of individually sequestered or discretely identifiable cells includes a nucleic acid vector or nucleic acid insert capable of expressing the one or more exogenous polynucleotides. Optionally, the population of individually sequestered or discretely identifiable cells expresses the one or more exogenous polynucleotides.

In embodiments, the one or more exogenous polynucleotides include a gRNA. Optionally, one or more exogenous polynucleotides are gRNAs.

In one embodiment, the method further includes comparing identities and levels of target transcripts and exogenous polynucleotides in the population of individually sequestered or discretely identifiable cells to identify exogenous polynucleotide-mediated gene perturbations in individual cells of the population of cells.

In embodiments, the population of individually sequestered or discretely identifiable cells is a population of individually sequestered or discretely identifiable mammalian cells. Optionally, the population of individually sequestered or discretely identifiable cells is a population of individually sequestered or discretely identifiable mammalian cell line cells. Optionally, the population of individually sequestered or discretely identifiable cells is a population of individually sequestered or discretely identifiable U937 lymphoma cell line cells. In some embodiments, the population of individually sequestered or discretely identifiable cells is a population of cells capable of acting as cellular factories (e.g., Chinese Hamster Ovary (CHO) cells, Human Embryonic Kidney (HEK, i.e., HEK293) cells, etc.) that can be further engineered for a specialized function via use of Stitch-seq. In other embodiments, the population of individually sequestered or discretely identifiable cells is a population of cells that reflect specific biology of interest, optionally utilized with Stitch-seq to understand relevant biology of such cells. In some embodiments, the population of individually sequestered or discretely identifiable cells is a population of primary cells.

In embodiments, the population of individually sequestered or discretely identifiable cells is a population of individually sequestered or discretely identifiable non-mammalian cells. Optionally, the population of individually sequestered or discretely identifiable cells is a population of microbial cells. In certain embodiments, the population of individually sequestered or discretely identifiable cells is a population of plant, bacteria and/or yeast cells. Optionally, the population of plant cells is a population of plant cells in suspension (i.e., a plant cell suspension culture).

In certain embodiments, the population of droplets or emulsions includes water-in-oil emulsions. Optionally, the oil is an immiscible oil. Optionally, the oil includes at least one fluorosurfactant. Optionally, the fluorosurfactant is a block copolymer consisting of one or more perfluorinated polyether (PFPE) blocks and one or more polyethylene glycol (PEG) blocks. Alternatively, the fluorosurfactant is a triblock copolymer consisting of a PEG center block covalently bound to two PFPE blocks by amide linking groups.

In embodiments, the population of droplets or emulsions has mean droplet or emulsion volumes of between about 10 pL and about 1 nL per individual droplet. In some embodiments, the population of droplets or emulsions has mean droplet or emulsion volumes of between about 80 pL and about 1.2 nL. In certain embodiments, the population of droplets or emulsions has mean droplet or emulsion volumes of between about 10 pL and about 80 pL. Optionally, the population of droplets or emulsions has mean droplet or emulsion volumes of between about 20 pL and about 80 pL. Optionally, the population of droplets or emulsions has mean droplet or emulsion volumes of between about 20 pL and about 60 pL. In other embodiments, the population of droplets or emulsions has mean droplet or emulsion volumes of between about 10 pL and about 20 pL, between about 20 pL and about 40 pL, or between about 40 pL and about 80 pL. In certain embodiments, the population of droplets or emulsions has mean droplet or emulsion volumes of between about 0.5 pL and about 10 pL. Optionally, the population of droplets or emulsions has mean droplet or emulsion volumes of between about 2 pL and about 5 pL. Optionally, the population of droplets or emulsions has mean droplet or emulsion volumes of about 3 pL or about 4 pL.

In some embodiments, the population of droplets has mean droplet sizes of between about 20 microns and about 200 microns in diameter per individual droplet. Optionally, the population of droplets has mean droplet sizes of between about 90 microns and about 150 microns in diameter per individual droplet. Optionally, the population of droplets has mean droplet sizes of between about 120 microns and about 145 microns in diameter per individual droplet, optionally about 135 microns in diameter per individual droplet. In other embodiments, the population of droplets has mean droplet sizes of between about 20 microns and about 90 microns in diameter per individual droplet. Optionally, the population of droplets has mean droplet sizes of between about 20 microns and about 70 microns in diameter per individual droplet. Optionally, the population of droplets has mean droplet sizes of between about 20 microns and about 50 microns in diameter per individual droplet.

In embodiments, the polynucleotide-guided protein is a polynucleotide-guided nuclease or a nuclease-dead functional variant thereof. In some embodiments, the polynucleotide-guided protein is a Cas enzyme or is RISC. Optionally, the Cas enzyme is a Cas9 or Cas13a enzyme. In embodiments, the Cas enzyme is dCAS9VPR or dCAS9-KRAB.

In certain embodiments, the nucleic acid amplification reagents include reverse transcriptase, a DNA polymerase, and one or more of the following types of primers: poly-T-tailed oligonucleotide primers, primers for specific amplification of the one or more exogenous polynucleotides capable of interacting with a polynucleotide-guided protein (or expressed polynucleotide proxy therefor), and/or primers for targeted transcript of interest amplification. In embodiments, the DNA polymerase is a thermostable DNA polymerase that enables PCR. Optionally, the thermostable DNA polymerase is a Taq DNA polymerase, e.g., AmpliTaq.

In some embodiments, the first pair of oligonucleotide primers amplifies a gRNA or RNAi agent sequence. Optionally, the gRNA or RNAi agent sequence is a component of a gRNA and/or RNAi agent library. In embodiments, the gRNA and/or RNAi agent library contains between 40 and 500,000 or more gRNAs and/or RNAi agents.

In embodiments, the first pair of oligonucleotide primers amplifies a nucleic acid sequence that identifies expression of a plurality of gRNAs. Optionally, the plurality of gRNAs and the sequence that identifies expression of the plurality of gRNAs are contained on a single vector. Optionally, the plurality of gRNAs includes three or more, four or more, five or more, or between five and twenty gRNAs. Optionally, the plurality of gRNAs includes ten to twenty gRNAs. Optionally, the single vector is a plasmid.

In some embodiments, the one or more target transcripts is capable of defining a cellular differentiation state, a cellular activation state, a cellular stress response state, and/or a cellular homeostatic state.

In certain embodiments, the one or more target transcripts include one or more of IRF3, DNA JC13, STINGI, TBK1 and TCF7. In some embodiments, the one or more target transcripts include one or more interferon stimulated genes (ISGs)—e.g., ADAR1, ISG15, USP18, STING, MDA5, PKR, EIF2a, ATF4, IRF9, RIG1, TBK1, IRF3, PD-L1, as well as combinations thereof.

In embodiments, the one or more target transcripts include a panel of transcripts for assessment of T-cell activation and/or differentiation status. Optionally, the panel of transcripts includes one or more T-cell receptor (TCR) and/or cluster of differentiation molecule (e.g., CD4, CD8, CD28, etc.) transcripts. Optionally, T-cells are identified to have a differentiation status that is naïve, memory, activated or exhausted.

In some embodiments, the one or more target transcripts include a panel of transcripts for assessment of B-cell activation and differentiation status. Optionally, the panel of transcripts includes B-cell receptor (BCR) transcripts. Optionally, B-cells are identified as having a differentiation status of naïve, memory, activated or plasmoblast.

In embodiments, the one or more target transcripts include a plurality of target transcripts, where individual droplets, hydrogel elements, microfluidic chip chambers, or array elements of the plurality of droplets, hydrogel elements, microfluidic chip chambers, or array elements include respective pairs of oligonucleotide primers for amplifying each target transcript of the plurality of target transcripts. Optionally, each of the respective pairs of oligonucleotide primers is designed for fusion by overlap extension of the target transcript amplicon with the amplicon of the first pair of oligonucleotide primers (e.g., the gRNA or gRNA identifying sequence-containing amplicon). Optionally, the plurality of target transcripts is multiplexed. Optionally, fusion of one or more target transcript amplicons with an associated gRNA amplicon occurs via intervening fusions with other target transcript amplicons within the individual droplet, hydrogel element, microfluidic chip chamber, or array element. E.g., not only can target transcripts be multiplexed within a droplet, hydrogel element, microfluidic chip chamber, or array element, but multiplexed target transcript amplicons within a droplet, hydrogel element, microfluidic chip chamber, or array element can also have primers designed such that the transcripts are joined in series with one another via fusion of multiple overlap extensions—in embodiments, such extended chimeric amplicons can be sequenced using long read sequencing (LRS) methods to resolve all such transcripts, together with associated gRNA sequences.

In certain embodiments, the individually sequestered or discretely identifiable cell is lysed by heating (e.g., during amplification) and/or by chemical means. Optionally, the individually sequestered or discretely identifiable cell is contacted with a Betaine solution (4 M, Sigma-Aldrich). Optionally, the individually sequestered or discretely identifiable cell is lysed while a population of droplets (e.g., droplet encapsulation of the individually sequestered cells) is being prepared.

In embodiments, the population of individually sequestered or discretely identifiable cells does not include microbeads.

In some embodiments, recovering fused amplicons from the population of individually sequestered or discretely identifiable cells (e.g., droplet-encapsulated cells) involves breaking open a population of droplets or emulsions. Optionally, breaking open the population of droplets or emulsions involves contacting the population of droplets or emulsions with a reagent that destabilizes the oil-water interface of the droplets or emulsions. Optionally, the reagent that destabilizes the oil-water interface is a large volume of high-salt solution. Optionally, the reagent that destabilizes the oil-water interface is a large volume (e.g., 30 mL) of perfluorooctanol (PFO) in 6×SSC. Alternatively, the reagent that destabilizes the oil-water interface is a small volume (e.g., 200 μL) of 20% PFO, optionally in HFE-7500 3M™ Novec™ engineered fluid. In certain embodiments, recovering fused amplicons from the population of individually sequestered or discretely identifiable cells (e.g., droplet-encapsulated cells) involves separation of a fused amplicon-containing aqueous phase from an oil phase. Optionally, such separation involves addition of Tris-EDTA (TE) buffer and chloroform, and performance of centrifugation (see Bio-Rad® QX200 Droplet Digital PCR System>Documents>6407: Droplet Digital PCR Applications Guide>pages 101-102 Amplicon Recovery from Droplets).

In embodiments, obtaining sequence from the fused amplicons includes use of a next-generation sequencing (NGS) method. Optionally, a paired-end NGS method is employed. Optionally, a bead-based paired-end NGS method is used, e.g., MiSeq®, NextSeq, or HiSeq®.

In some embodiments, obtaining sequences from the fused amplicons involves use of a long read sequencing (LRS) method.

In certain embodiments, fused amplicon sequence data are obtained and then used to assemble a matrix of digital gene-expression measurements including counts of each expressed target transcript detected in each cell. Optionally, further analysis is then performed, e.g., to resolve gRNA-mediated transcriptional modulations at the single cell (or single droplet) level.

In embodiments, paired transcript and exogenous polynucleotide (e.g., gRNA, RNAi agent or other exogenous polynucleotide) sequences of fused amplicons are obtained for at least 10,000 individual cells. Optionally, paired transcript and exogenous polynucleotide sequences of fused amplicons are obtained for at least 100,000 individual cells. Optionally, paired transcript and exogenous polynucleotide sequences of fused amplicons are obtained for about 1,000,000 or more individual cells.

In certain embodiments, the gene perturbation effects of at least 1,000 different exogenous polynucleotides are assessed in the population of individually sequestered or discretely identifiable cells.

In some embodiments, the plurality of oligonucleotides further includes a third pair of oligonucleotide primers for amplifying an exogenous polynucleotide or a second target transcript of the individually sequestered or discretely identifiable cell. Optionally, three or more distinct nucleic acid sequences are fused in performing a method of the instant disclosure.

An additional aspect of the disclosure provides a droplet or emulsion having a fused amplicon including a target transcript amplicon joined with an exogenous polynucleotide or an exogenous polynucleotide identifier sequence amplicon, where the fused amplicon is formed by overlap extension and where the optional exogenous polynucleotide identifier sequence is an expressed sequence that indicates the presence in the droplet of a specific combination of exogenous polynucleotides.

Another aspect of the instant disclosure provides a method for identifying within a population of individually sequestered or discretely identifiable cells one or more polynucleotide-tagged polypeptides or one or more polynucleotide tag-associated polypeptides and one or more target transcripts in an individual sequestered or discretely identifiable cell, the method involving (a) preparing or providing a population of individually sequestered or discretely identifiable cells, where a plurality of individually sequestered or discretely identifiable cells harbors or expresses a polynucleotide-tagged polypeptide or expresses a polynucleotide tag that indicates expression of one or more tag-associated polypeptides in the cell and a plurality of the individually sequestered or discretely identifiable cells are contacted with nucleic acid amplification reagents and a plurality of oligonucleotides including: (i) a first pair of oligonucleotide primers for amplifying a tag of the polynucleotide-tagged polypeptide or the polynucleotide tag that indicates the presence or expression of the one or more associated polypeptides in the individually sequestered or discretely identifiable cell; and (ii) a second pair of oligonucleotide primers for amplifying a target transcript of the individually sequestered or discretely identifiable cell, where the first pair of oligonucleotide primers possesses a primer having a 5′-terminal region of sequence that is the same or complementary to a 5′-terminal region of sequence of a primer of the second pair of oligonucleotide primers, where the 5′-terminal region that is the same or complementary between the first pair of oligonucleotide primers and the second pair of oligonucleotide primers is of sufficient length to allow for amplification-mediated joining of an amplicon of the first pair of oligonucleotide primers and an amplicon of the second pair of oligonucleotide primers into a fused amplicon, where the individually sequestered or discretely identifiable cell is lysed to render contents of the cell accessible (e.g., to enzymes and/or oligonucleotides) in a manner that maintains the sequestering or discrete identification of the lysed cell contents; (b) performing polymerase-mediated primer extension and optionally thermal cycling upon the population of lysed cell contents under conditions suitable for generating fused amplicons comprising the amplicon of the first pair of oligonucleotide primers and the amplicon of the second pair of oligonucleotide primers by overlap extension, thereby generating fused amplicons within the population of individually sequestered or discretely identifiable lysed cell contents; (c) recovering fused amplicons from the population of lysed cell contents; and (d) obtaining sequence information from the fused amplicons using a sequencing method capable of obtaining sequences from both ends of individual fused amplicon sequences and identifying as a pair the sequences obtained from both ends of the same individual fused amplicon, thereby identifying in the population of individually sequestered or discretely identifiable cells one or more target transcripts and one or more polynucleotide-tagged polypeptides or expressed polynucleotide tag-associated polypeptides within the individually sequestered or discretely identifiable cell.

In some embodiments, the polypeptides of the one or more polynucleotide-tagged polypeptides or one or more polynucleotide tag-associated polypeptides include one or more transcription factors.

In embodiments, the polypeptides of the one or more polynucleotide-tagged polypeptides or one or more polynucleotide tag-associated polypeptides include one or more protein variants. Optionally, the polynucleotides are of a protein variant library.

In certain embodiments, the polypeptides of the one or more polynucleotide-tagged polypeptides or one or more polynucleotide tag-associated polypeptides are members of and/or are derived from one or more protein libraries.

In an additional aspect, the instant disclosure provides a method for identifying within a population of oil droplet-encapsulated or emulsion-encapsulated cells one or more target transcripts and one or more exogenous polynucleotides capable of interacting with a polynucleotide-guided protein as co-expressed in an individual droplet-encapsulated or emulsion-encapsulated cell, the method including: (a) preparing or providing a population of droplets or emulsions, where a plurality of droplets or emulsions includes: an individual droplet-encapsulated or emulsion-encapsulated cell harboring or expressing a polynucleotide-guided protein capable of interacting with the one or more exogenous polynucleotides, where the individual droplet-encapsulated or emulsion-encapsulated cell also expresses one or more exogenous polynucleotides capable of interacting with a polynucleotide-guided protein or includes a nucleic acid vector capable of expressing one or more exogenous polynucleotides capable of interacting with a polynucleotide-guided protein; nucleic acid amplification reagents (e.g., RT-PCR reagents); and a plurality of oligonucleotides including: (i) a first pair of oligonucleotide primers for amplifying an exogenous polynucleotide capable of interacting with a polynucleotide-guided protein or a nucleic acid sequence that identifies expression of one or more exogenous polynucleotides capable of interacting with a polynucleotide-guided protein in the individual droplet-encapsulated or emulsion-encapsulated cell; and (ii) a second pair of oligonucleotide primers for amplifying a target transcript of the individual droplet-encapsulated or emulsion-encapsulated cell, where the first pair of oligonucleotide primers possesses a primer having a 5′-terminal region of sequence that is the same or complementary to a 5′-terminal region of sequence of a primer of the second pair of oligonucleotide primers, where the 5′-terminal region that is the same or complementary between the first pair of oligonucleotide primers and the second pair of oligonucleotide primers is of sufficient length to allow for amplification-mediated joining of an amplicon of the first pair of oligonucleotide primers and an amplicon of the second pair of oligonucleotide primers into a fused amplicon, where the individual droplet-encapsulated or emulsion-encapsulated cell is lysed within the population of droplets or emulsions; (b) performing polymerase-mediated primer extension and optionally thermal cycling upon the population of droplets or emulsions under conditions suitable for generating fused amplicons including the amplicon of the first pair of oligonucleotide primers and the amplicon of the second pair of oligonucleotide primers by overlap extension, thereby generating fused amplicons within the individual droplet or emulsion; (c) recovering fused amplicons from the population of droplets or emulsions; and (d) obtaining sequence information from the fused amplicons using a sequencing method capable of obtaining sequences from both ends of individual fused amplicon sequences and identifying as a pair said sequences obtained from both ends of the same individual fused amplicon, thereby identifying in the population of droplet-encapsulated or emulsion-encapsulated cells one or more target transcripts and one or more exogenous polynucleotides capable of interacting with a polynucleotide-guided protein as co-expressed within the individual droplet-encapsulated or emulsion-encapsulated cell.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

By “control” or “reference” is meant a standard of comparison. Methods to select and test control samples are within the ability of those in the art. Determination of statistical significance is within the ability of those skilled in the art, e.g., the number of standard deviations from the mean that constitute a positive result.

As used herein, the term “different”, when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more nucleic acids can have target nucleotide sequence portions that are different for the two or more molecules while also having a universal sequence portion that is the same on the two or more molecules.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “polynucleotide-guided protein” refers to any protein for which a functional activity of the protein is modulated (e.g., activated) by contact with a polynucleotide sequence. Exemplary polynucleotide-guided proteins include polynucleotide guided enzymes and/or nucleases, including, without limitation, Cas9, Cas13 and/or other Cas enzyme variants, as well as RNA-induced silencing complex (RISC), among others.

As used herein, the term “guide RNA” or “gRNA” refers to a CRISPR system guide RNA. Guide RNAs that exist as a single RNA molecule may be referred to as single-guide RNAs (sgRNAs), though “gRNA” is also used to refer to guide RNAs that exist as either single molecules or as a complex of two or more molecules. Typically, gRNAs that exist as a single RNA species comprise two domains: (1) a domain that shares homology to a target nucleic acid (i.e., directs binding of a Cas9 complex to the target); and (2) a domain that binds a Cas9 domain. In some embodiments, domain (2) corresponds to a sequence known as a tracrRNA and comprises a stem-loop structure. In some embodiments, domain (2) is identical or homologous to a tracrRNA as provided in Jinek et al. Science 337:816-821 (2012), the entire contents of which is incorporated herein by reference. Other examples of gRNAs (e.g., those including domain 2) can be found in International Patent Application PCT/US2014/054252, filed Sep. 5, 2014, entitled “Switchable Cas9 Nucleases And Uses Thereof,” and International Patent Application PCT/US2014/054247, filed Sep. 5, 2014, entitled “Delivery System For Functional Nucleases,” the entire contents of each are hereby incorporated by reference in their entirety. In some embodiments, a gRNA comprises two or more of domains (1) and (2), and may be referred to as an “extended gRNA.” For example, an extended gRNA will bind two or more Cas9 domains and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example, Cas9 (also known as Csn1) from Streptococcus pyogenes (see, e.g., “Complete genome sequence of an Ml strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai Fi. S., Lin S. P, Qian Y., Jia Fi. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Fiauer M., Doudna J. A., Charpentier E. Science 337:816-821 (2012), the entire contents of each of which are incorporated herein by reference).

Because RNA-programmable nucleases (e.g., Cas9) use RNA:DNA hybridization to target DNA cleavage sites, these proteins are able to target, in principle, any sequence specified by the guide RNA. Methods of using RNA-programmable nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature Biotechnology 31, 227-229 (2013); Jinek, M. et al. RNA-programmed genome editing in human cells. eLife 2, e00471 (2013); Dicarlo, J. E. et al. Genome engineering in Saccharomyces cerevisiae using CRISPR-Cas systems. Nucleic Acids Research (2013); Jiang, W. et al. RNA-guided editing of bacterial genomes using CRISPR-Cas systems. Nature Biotechnology 31, 233-239 (2013); the entire contents of each of which are incorporated herein by reference).

In general, a “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr mate sequence (encompassing a “direct repeat” and a tracrRNAprocessed partial direct repeat in the context of an endogenous CRISPR system), a guide sequence (also referred to as a “spacer” in the context of an endogenous CRISPR system), or other sequences and transcripts from a CRISPR locus. The tracrRNA of the system is complementary (fully or partially) to the tracr mate sequence present on the guide RNA.

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casnl nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids.

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template, such methods including those disclosed herein (which involve polymerase extension and exonuclease- and/or primer-mediated strand displacement), as well as art-recognized amplification methods including, for example, polymerase extension, polymerase chain reaction (PCR), rolling circle amplification (RCA), multiple displacement amplification (MDA), ligation extension, or ligation chain reaction. An amplicon can be a nucleic acid molecule having a single copy of a particular nucleotide sequence (e.g., a PCR product) or multiple copies of the nucleotide sequence (e.g., a concatameric product of RCA). A first amplicon of a target nucleic acid is typically a complementary copy. Subsequent amplicons are copies that are created, after generation of the first amplicon, from the target nucleic acid or from the first amplicon (the template nucleic acid or its complement, noting that reference to a complement nucleic acid can refer to the complement of a subsequence of a template nucleic acid, not necessarily to a sequence that is fully complementary with the template nucleic acid across the entire length of the template nucleic acid—e.g., the initial complementary sequence of an amplification method as disclosed herein will generally be of shorter length than the template nucleic acid, and the complementary sequence of the template nucleic acid may also include one or more mutations yet still allow for the methods of the instant disclosure to proceed effectively, with introduction of such mutations depending upon the fidelity of the polymerase employed and the effects of chance). A subsequent amplicon can have a sequence that is substantially complementary to the target nucleic acid or substantially identical to the target nucleic acid.

As used herein, the term “extend,” when used in reference to a nucleic acid, is intended to mean addition of at least one nucleotide or oligonucleotide to the nucleic acid. In particular embodiments, one or more nucleotides can be added to the 3′ end of a nucleic acid, for example, via polymerase catalysis (e.g., DNA polymerase, RNA polymerase or reverse transcriptase). Chemical or enzymatic methods can be used to add one or more nucleotide to the 3′ or 5′ end of a nucleic acid. One or more oligonucleotides can be added to the 3′ or 5′ end of a nucleic acid, for example, via chemical or enzymatic (e.g., ligase catalysis) methods. A nucleic acid can be extended in a template directed manner, whereby the product of extension is complementary to a template nucleic acid that is hybridized to the nucleic acid that is extended.

As used herein, the term “reverse transcriptase” refers to an enzyme used to generate complementary DNA (cDNA) from an RNA template. Without limitation, exemplary reverse transcriptases (RTs) expressly contemplated for use with the instant disclosure include RTX (RT “xenopolymerase”—Ellefson et al. Science 352: 1590-93)), AMV, M-MLV, and ProScript® RT. In certain embodiments, Taq DNA polymerase is also an exemplary reverse transcriptase (per Bhadra et al. Biochemistry 59: 4638-4645).

As used herein, “amplify”, “amplifying” or “amplification reaction” and their derivatives, refer generally to any action or process whereby at least a portion of a nucleic acid molecule is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. Amplification optionally includes linear or exponential replication of a nucleic acid molecule. In certain embodiments featured herein, such amplification can be performed using isothermal conditions (isothermal amplification). In other embodiments, such amplification can include thermocycling. In some embodiments, the amplification is a multiplex amplification that includes the simultaneous amplification of a plurality of target sequences in a single amplification reaction. The amplification reaction can include any of the amplification processes known to one of ordinary skill in the art. In certain embodiments featured herein, the amplification reaction includes a combination of polymerase, exonuclease and nucleic acid primers (optionally, modified nucleic acid primers). In some embodiments, an amplification reaction can include polymerase chain reaction (PCR) amplifying one or more nucleic acid sequences. Amplification can be linear or exponential. In some embodiments, the amplification conditions can include isothermal conditions or alternatively can include thermocycling conditions, or a combination of isothermal and thermocycling conditions. In some embodiments, the conditions suitable for amplifying one or more nucleic acid sequences include polymerase chain reaction (PCR) conditions. Typically, the amplification conditions refer to a reaction mixture that is sufficient to amplify nucleic acids such as one or more target sequences flanked by a universal sequence, or to amplify an amplified target sequence ligated to one or more adapters. Generally, the amplification conditions include a catalyst for amplification or for nucleic acid synthesis, for example a polymerase; a primer that possesses some degree of complementarity to the nucleic acid to be amplified; and nucleotides, such as deoxyribonucleotide triphosphates and ribononucleic triphosphates to promote extension of the primer once hybridized to the nucleic acid. The amplification conditions can require hybridization or annealing of a primer to a nucleic acid, extension of the primer and a strand displacement step in which the extended primer is separated from the nucleic acid sequence undergoing amplification. As used herein, “amplified target sequences” and its derivatives, refers generally to a nucleic acid sequence produced by the amplifying the target sequences using target-specific primers and the methods provided herein. The amplified target sequences may be either of the same sense (i.e., the positive strand) or antisense (i.e., the negative strand) with respect to the target sequences.

As used herein, the term “target nucleic acid” refers to a nucleic acid that is desired to be amplified in a nucleic acid amplification reaction. For example, the target nucleic acid comprises a nucleic acid template (e.g., a transcript of interest).

As used herein, the term “DNA polymerase” refers to an enzyme that synthesizes a DNA strand de novo using a nucleic acid strand as a template. DNA polymerase uses an existing DNA or RNA as the template for DNA synthesis and catalyzes the polymerization of deoxyribonucleotides alongside the template strand, which it reads. The newly synthesized DNA strand is complementary to the template strand. DNA polymerase can add free nucleotides only to the 3′-hydroxyl end of the newly forming strand. It synthesizes oligonucleotides via transfer of a nucleoside monophosphate from a deoxyribonucleoside triphosphate (dNTP) to the 3′-hydroxyl group of a growing oligonucleotide chain. This results in elongation of the new strand in a 5′-3′ direction. Since DNA polymerase can only add a nucleotide onto a pre-existing 3′—OH group, to begin a DNA synthesis reaction, the DNA polymerase needs a primer to which it can add the first nucleotide. Suitable primers comprise oligonucleotides of DNA or RNA. A DNA polymerase employed herein may be a naturally occurring DNA polymerase or a variant of a natural enzyme having the above-mentioned activity.

As used herein, the term “plasmid” refers to an extra-chromosomal nucleic acid that is separate from a chromosomal nucleic acid. A plasmid DNA may be capable of replicating independently of the chromosomal nucleic acid (chromosomal DNA) in a cell. Plasmid DNA is often circular and double-stranded.

As used herein, the terms “nucleic acid” and “nucleotide” are intended to be consistent with their use in the art and to include naturally occurring species or functional analogs thereof. Particularly useful functional analogs of nucleic acids are capable of hybridizing to a nucleic acid in a sequence specific fashion or capable of being used as a template for replication of a particular nucleotide sequence.

As used herein, the “percent identity” of given nucleic acid sequences describes the similarity of two or more sequences, as determined by sequence alignment, including the introduction of gaps for optimal alignment where necessary. When a position in one sequence is occupied by the same residue as the corresponding position in another sequence, the molecules are identical at that position. The percent identity between any two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between any two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, a gapped alignment is employed wherein the alignment is optimized by introducing appropriate gaps, and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, a global alignment the alignment is optimized by introducing appropriate gaps, and percent identity is determined over the entire length of the sequences aligned. (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package.

Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g., found in deoxyribonucleic acid (DNA)) or a ribose sugar (e.g., found in ribonucleic acid (RNA)). A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine, thymine, cytosine or guanine and a ribonucleic acid can have one or more bases selected from the group consisting of uracil, adenine, cytosine or guanine. Useful non-native bases that can be included in a nucleic acid or nucleotide are known in the art. The terms “probe” or “target,” when used in reference to a nucleic acid or sequence of a nucleic acid, are intended as semantic identifiers for the nucleic acid or sequence in the context of a method or composition set forth herein and does not necessarily limit the structure or function of the nucleic acid or sequence beyond what is otherwise explicitly indicated.

As used herein, the term “primer” and its derivatives refer generally to any nucleic acid that can hybridize to a target sequence of interest. Typically, the primer functions as a substrate onto which nucleotides can be polymerized by a polymerase or to which a nucleotide sequence such as an index can be ligated; in some embodiments, however, the primer can become incorporated into the synthesized nucleic acid strand and provide a site to which another primer can hybridize to prime synthesis of a new strand that is complementary to the synthesized nucleic acid molecule. The primer can include any combination of nucleotides or analogs thereof. In some embodiments, the primer is a single-stranded oligonucleotide or polynucleotide. The terms “polynucleotide” and “oligonucleotide” are used interchangeably herein to refer to a polymeric form of nucleotides of any length, and may include ribonucleotides, deoxyribonucleotides, analogs thereof, or mixtures thereof. The terms should be understood to include, as equivalents, analogs of either DNA, RNA, or cDNA and double stranded polynucleotides. The term as used herein also encompasses cDNA, that is complementary or copy DNA produced from an RNA template, for example by the action of reverse transcriptase. This term refers only to the primary structure of the molecule.

As used herein, the term “next-generation sequencing” or “NGS” can refer to sequencing technologies that have the capacity to sequence polynucleotides at speeds that were unprecedented using conventional sequencing methods (e.g., standard Sanger or Maxam-Gilbert sequencing methods). These unprecedented speeds are achieved by performing and reading out thousands to millions of sequencing reactions in parallel. NGS sequencing platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina™); SOLiD™ technology (Applied Biosystems); Ion semiconductor sequencing (Ion Torrent™); and DNA nanoball sequencing (Complete Genomics). Descriptions of certain NGS platforms can be found in the following: Shendure, er al., “Next-generation DNA sequencing,” Nature, 2008, vol. 26, No. 10, 135-1 145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11 (3):333-43; and Zhang et al., “The impact of next-generation sequencing on genomics”, J Genet Genomics, 201, 38(3): 95-109. In certain embodiments, the sequencing parameters of NGS approaches can be modified to allow the instant methods to obtain extended average read lengths during sequencing. In embodiments, long read sequencing (LRS) approaches can also be employed to obtain average read lengths that exceed those of current high-throughput NGS approaches—e.g., such LRS approaches can achieve individual read lengths approaching a megabase or more in certain applications, though generally with lower throughput than the above-described NGS methods. Exemplary forms of long read sequencing include, without limitation, single molecule real time sequencing (SMRT; based on the properties of zero-mode waveguides; signals are in the form of fluorescent light emission from each nucleotide incorporated by a DNA polymerase bound to the bottom of the zL well; developed by PacBio® and used in, e.g., single-cell isoform RNA sequencing (ScISOr-seq)) and nanopore sequencing (which involves passing a DNA molecule through a nanoscale pore structure and then measuring changes in electrical field surrounding the pore, developed by Oxford Nanopore).

As used herein, the term “poly T or poly A,” when used in reference to a nucleic acid sequence, is intended to mean a series of two or more thiamine (T) or adenine (A) bases, respectively. A poly T or poly A can include at least about 2, 5, 8, 10, 12, 15, 18, 20 or more of the T or A bases, respectively. Alternatively or additionally, a poly T or poly A can include at most about, 30, 20, 18, 15, 12, 10, 8, 5 or 2 of the T or A bases, respectively.

As used herein, the term “subject” includes humans and animals, including mammals (e.g., mice, rats, pigs, cats, dogs, and horses), as well as fish, birds, reptiles, insects, mollusks, and other animals. In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

The embodiments set forth below and recited in the claims can be understood in view of the above definitions.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIG. 1 shows a schematic of an exemplary process of the disclosure. Cells with a perturbation of interest were subjected to single-cell emulsion, with encapsulated cells then lysed via heat treatment. The lysed contents of encapsulated single cells (in certain examples, droplet-encapsulated cells from the CROP-seq process were used, cells of which express Cas enzyme and guide RNA(s)) were subjected to reverse transcription and PCR amplification of target transcript sequences (here, three target transcripts are shown, as well as a perturbation nucleic acid), while reverse transcription and PCR amplification also amplified a perturbation nucleic acid (for example, perturbation mRNAs, expressed gRNAs, etc.). Overlap extension primers are employed during generation of initial amplicons, with 5′ tail sequences that are the same or complementary included on at least one primer of each pair of amplification primers. Such 5′-tails promote the overlap extension process to occur, ultimately resulting in fused, extended amplicons that pair the perturbation nucleic acid with target transcript(s), within the same amplicon. Emulsions (droplets) were then broken open, and fused amplicons derived from the population of droplets were then pooled and cleaned/isolated as a nucleic acid library, in preparation for sequencing. In further preparation for sequencing of fused amplicons, paired amplification primers tailed with adapter sequences compatible with an Illumina® NGS platform were employed, to add sequencing adapters to the ends of fused amplicons (still including sufficient target transcript sequence for discrete identification of target transcripts during sequencing and also including paired perturbation nucleic acid (e.g., gRNA) sequences). Nested amplification and library preparation is thereby performed. Paired-end NGS sequencing is then performed upon adapter-presenting amplicons, resulting in identification of perturbation nucleic acid-target transcript associations (at the single droplet/single perturbation nucleic acid level of resolution), in a robustly parallel, high-throughput manner, that does not require the microbeads used, e.g., in previous DROP-seq and/or CROP-seq implementations.

FIG. 2 shows pilot results for design and use of overlap extension (OE) primers to amplify target transcripts (genes of interest) in U937 cells. Target transcript amplifying/OE primers for IRF3 (“i1” and “i2”), DNA JC13 (“j1” and “j2”), STINGI (“s1” and “s2”), TBK1 (“tb1” and “tb2”) and TCF7 (“tc1” and “tc2”) were assessed, in two distinct sets, both in the presence and absence of housekeeping genes (housekeeping genes have been described to sequester gRNA during stitching). “g1” indicates GAPDH; “a2” indicates Actin. An initial stitch (via OE) reaction was performed in multiplex, followed by individual nested PCRs. Primer optimization was achieved via normal PCR, standard dilution and stitching (via OE). As seen in the currently exemplified pilot phase, transcripts of different levels were observed.

FIG. 3 shows exemplary droplets of the current disclosure. Cells were treated with trypsin for 2 min and a Betaine-containing PCR reagent solution was only added immediately in advance of dropletization. With current droplet sizes, yields were up to 100k cells/mL of droplets; however, available reductions in droplet size are contemplated to provide yields of a million or more cells/mL. PCR is performed upon droplets of the instant disclosure in a manner similar to DROP-seq, and the DROP-seq dropletizer is currently used for droplet production—specifically, the dropletizer takes in cells and reagents, and droplets, once formed, are of approximately uniform size (as shown), have minimal multiplets (droplets with more than one cell, as shown with red arrows), and are stable enough to allow for PCR to be performed in droplets.

FIG. 4 shows PCR products obtained from performing nested PCR amplification employing Stitch-seq upon U937 cells lysed within single cell droplets. U937 cells were incorporated into single cell droplets (dropletized) at levels of 100k cells/mL. U937 cell-containing droplets were then subjected to multiplex droplet-based Stitch PCR, which linked cognate gRNAs to GAPDH, IRF3, TBK1, and STINGI, respectfully. Gel electrophoresis was then performed to visualize fused PCR amplicons after the nested PCR.

FIG. 5 shows quantitative benchmarking of Stitch-seq. The percentage of reads (log scale) that aligned to each synthetic target gene (at different initial concentrations of synthetic target gene) for a range of gRNA concentrations were compared to the expected proportion of reads given the initial concentration. A highly correlated R² value of 0.984 was observed for the 16.2 nM gRNA concentration.

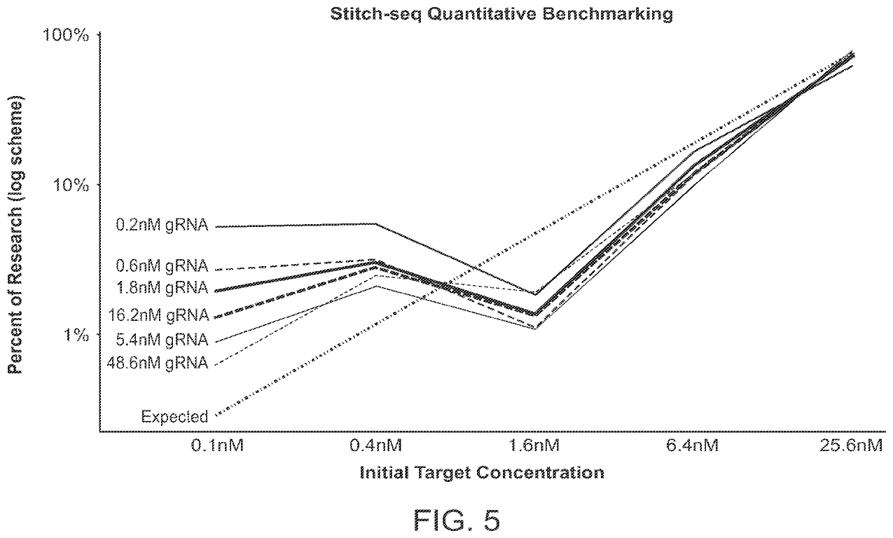

FIG. 6 demonstrates droplet stability in exemplified Stitch-seq reactions. At left, complete Stitch-seq reaction conditions in oil droplets were imaged before thermocycling. At right, the droplet population after thermocycling for the Stitch PCR was imaged. The image comparison revealed robust droplet stability after thermocycling for the Stitch PCR.

FIG. 7 shows droplet mixing and reaction fidelity. Two engineered cell lines were dropletized, each stably expressing either reporter A or B. Unique parts of the transcripts (A1, A2, B1, B2) were amplified such that A1 or B1 could stitch to A2 or B2, each generating fragments of different sizes depending on what is amplifying (A1+A2, B1+B2, A1+B2, B1+A2). Lane 1 shows nested PCR product from a bulk Stitch PCR performed on cells containing reporter A (only A1+A2 possible). Lane 2 shows nested PCR product from a bulk Stitch PCR performed on cells containing reporter B, where only B1+B2 was possible. Lane 3 shows product of a nested PCR performed on product from the Stitch PCRs of conditions 1 and 2 to identify any crossover during the nested PCR. Lane 4 shows nested PCR product of cells containing reporter A and cells containing reporter B that were input into a bulk Stitch PCR together. Lane 5 shows nested PCR product from a droplet Stitch PCR performed on cells containing reporter A. Lane 6 shows nested PCR product from a droplet Stitch PCR performed on cells containing reporter B. Lane 7 shows nested PCR product from a droplet Stitch PCR of cells with reporter A dropletized separately from cells with reporter B. However, droplets were mixed for the Stitch PCR to identify droplet merging during the Stitch PCR. Lane 8 shows nested PCR product of a droplet Stitch PCR performed on cells with reporter A and cells with reporter B dropletized together for the Stitch PCR to identify doublets during dropletization. If there are no doublets, no droplet mixing during the Stitch PCR, and no cross-product amplification would occur during the nested PCR, it would result in only two bands. This is condition 3, A1+A2, B1+B2. If there are doublets/mixing/crossover, there will be 4 bands. This is condition 4, A1+A2, B1+B2, A1+B2, B1+A2. The gel image shows that there was no nested PCR crossover (condition 3), no droplet merging (condition 7), and minimal doublets (condition 8) throughout Stitch-seq, meaning that the droplets were successfully compartmentalizing each cell for Stitch PCR, and that Stitch-seq maintained fidelity of the reaction inputs.

### DETAILED DESCRIPTION OF THE INVENTION

The present disclosure is directed, at least in part, to discovery of a method for enhancing droplet-based assessment of gRNA-mediated transcriptional perturbations (such as those previously described in the CROP-seq process of Datlinger et al.), via application of overlap extension (OE)-mediated fusion of gRNA sequences with target transcripts during in-droplet amplification reactions, which is followed by bulk sequencing of fused amplicons in a manner that retains and identifies droplet-specific associations between gRNAs and target transcripts captured within such fused amplicons. Advantageously, the process of the instant disclosure can be applied to a wide range of exogenous nucleic acids (e.g., gRNAs, lineage barcodes, etc.), can be applied to any population of individually sequestered (e.g., droplet-encapsulated, hydrogel-contained or otherwise arrayed, e.g., distributed in a microwell array) and/or discretely identifiable (e.g., tagged) cells, and does not required co-encapsulation of cells and beads for assessing exogenous nucleic acid-mediated modulation of target transcripts, which allows for enhanced throughput over single cell transcriptome-monitoring approaches previously described in the art. Single cell transcriptional perturbation data is thereby obtained, for perturbations mediated by individually identifiable exogenous nucleic acids within a cell, at a scale that allows for tens of thousands to millions of cells to be surveyed in a single experiment, to detect exogenous nucleic acid-mediated transcriptional perturbations at the single cell level.

Traditional CRISPR screens only identify a change in cell fitness represented by a change in gRNA abundance and so are unable to characterize the phenotypic output of gene perturbation. Tracking phenotypic changes caused by genetic perturbations at the single-cell level, at high-throughput, as the instant disclosure provides, enables the systematic exploration of the genetic contributions to complex phenotypes associated with development and disease to be performed. CROP-seq is a well-known approach that combines traditional CRISPR screens and scRNA-seq to enable a single-cell transcriptomic output with perturbation resolution. However, such art-recognized screens have been severely limited by throughput, and so have been unable to simultaneously measure the effects of multiple perturbations on multiple gene targets.

To address the above-noted throughput limitations of droplet-based single cell transcriptome monitoring processes such as CROP-seq, in certain aspects, the instant disclosure provides a process (termed “Stitch-seq” herein) in which individual cells from a CROP-seq perturbation library are encapsulated in an oil emulsion and the mRNA transcripts of interest are stitched to the cell's cognate gRNA via overlap extension RT-PCR. The native pairing of the gRNA and several transcripts of interest at the single cell level allows for the coupling of targeted gene expression alterations associated with specific gRNA-mediated perturbations at significantly higher throughput than existing methods.

In certain embodiments, a set of perturbations can be performed on a population of naïve T-cells (CROP-seq) and the expression of genes related to T-cell differentiation can be analyzed using the current process, to quickly determine the effect of each perturbation on differentiation. Once perturbations are identified that drastically change the expression of genes of interest, further exploration can be performed.

By physically linking within individual droplets the gRNA with the expressed transcripts of interest, the process of the instant disclosure drastically simplifies the workflow for capturing perturbation effects on gene expression, at least by circumventing the use of beads during droplet manipulation processes, which thereby provides for large increases in screen scale. Accordingly, the instant disclosure provides for quick identification of perturbations that warrant more in-depth testing.

Various expressly contemplated components of certain methods and compositions of the instant disclosure are considered in additional detail below.

### CROP-Seq

Certain embodiments of the instant disclosure have adapted the CROP-seq process to provide for higher throughput assessment of gRNA-mediated cellular target transcript perturbations. CROP-Seq (also known as CRISP-seq and Perturb-seq) is a well-known approach that combines traditional CRISPR screens and scRNA-seq to enable a single-cell transcriptomic output with perturbation resolution (refer to Datlinger et al. Nature Methods. 14: 297-301). In CROP-seq, individual gRNAs of a gRNA library are integrated into cells using lentiviral vectors, and are then expressed within the cell, with expressed, active gRNAs as currently exemplified also having a 3′-UTR and poly-A tail, which allows for expressed gRNAs to be amplified using RT-PCR, in parallel, e.g., with amplification by RT-PCR of target transcripts.

CROP-seq specifically refers to a high-throughput method of performing single cell RNA sequencing (scRNA-seq) on pooled genetic perturbation screens (Adamson et al. Cell. 167 (7): 1867-1882; Dixit et al. Cell. 167 (7): 1853-1866; Datlinger et al. Nature Methods. 14 (3): 297-301). CROP-seq combines multiplexed CRISPR mediated gene inactivations with single cell RNA sequencing to assess comprehensive gene expression phenotypes for each perturbation. Inferring a gene's function by applying genetic perturbations to knock down or knock out a gene and studying the resulting phenotype is known as reverse genetics. CROP-seq is a reverse genetics approach that allows for the investigation of phenotypes at the level of the transcriptome, to elucidate gene functions in many cells, in a massively parallel fashion.

The CROP-seq protocol uses CRISPR technology to inactivate specific genes and DNA barcoding of each guide RNA to allow for all perturbations to be pooled together and later deconvoluted, with assignment of each phenotype to a specific guide RNA (Adamson et al. Cell. 167 (7): 1867-1882; Dixit et al. Cell. 167 (7): 1853-1866). Droplet-based microfluidics platforms (or other cell sorting and separating techniques) are used to isolate individual cells, and then scRNA-seq is performed to generate gene expression profiles for each cell. Upon completion of the protocol, bioinformatics analyses are conducted to associate each specific cell and perturbation with a transcriptomic profile that characterizes the consequences of inactivating each gene.

Pooled CRISPR libraries that enable gene inactivation can come in the form of either knockout or interference. Knockout libraries perturb genes through double stranded breaks that prompt the error prone non-homologous end joining repair pathway to introduce disruptive insertions or deletions. CRISPR interference (CRISPRi) on the other hand utilizes a catalytically inactive nuclease to physically block RNA polymerase, effectively preventing or halting transcription (Larson et al. Nature Protocols. 8 (11): 2180-2196). CROP-seq has been utilized with both the knockout and CRISPRi approaches in Dixit et al. and Adamson et al., respectively.

In CROP-seq, pooling all guide RNAs into a single screen tends to rely upon DNA barcodes that act as identifiers for each unique guide RNA. There are several commercially available pooled CRISPR libraries including the guide barcode library used in the study by Adamson et al. CRISPR libraries can also be custom made using tools for sgRNA design.

sgRNA expression vector design in CROP-seq employs lentiviral vectors for delivery, with such vectors including the following central components: promoter, restriction sites, primer binding sites, sgRNA, guide barcode, reporter gene, fluorescent gene (e.g., GFP, as vectors are often constructed to include a gene encoding a fluorescent protein, such that successfully transduced cells can be visually and quantitatively assessed by their expression), antibiotic resistance gene (similar to fluorescent markers, antibiotic resistance genes are often incorporated into vectors to allow for selection of successfully transduced cells), and a CRISPR-associated endonuclease (Cas9 or other CRISPR-associated endonucleases such as Cpf1 must be introduced to cells that do not endogenously express them. Due to the large size of these genes, a two-vector system can be used to express the endonuclease separately from the sgRNA expression vector (Shalem et al. Science. 343 (6166): 84-87).)

In CROP-seq, cells are typically transduced with a Multiplicity of Infection (MOI) of 0.4 to 0.6 lentiviral particles per cell to maximize the likelihood of obtaining the most amount of cells which contain a single guide RNA (Shalem et al. Science. 343 (6166): 84-87; Wang et al. Science. 343 (6166): 80-84). If the effects of simultaneous perturbations are of interest, a higher MOI may be applied to increase the amount of transduced cells with more than one guide RNA. Selection for successfully transduced cells is then performed using a fluorescence assay or an antibiotic assay, depending on the reporter gene used in the expression vector.

After successfully transduced cells have been selected for, isolation of single cells is needed to conduct scRNA-seq. CROP-seq has been performed using droplet-based technology for single cell isolation (Adamson et al. Cell. 167 (7): 1867-1882; Dixit et al. Cell. 167 (7): 1853-1866; Datlinger et al. Nature Methods. 14 (3): 297-301). Once cells have been isolated at the single cell level, reverse transcription, amplification and sequencing takes place to produce gene expression profiles for each cell. Many scRNA-seq approaches employ beads and incorporate unique molecular identifiers (UMIs) and cell barcodes during the reverse transcription step to index individual RNA molecules and cells, respectively. These additional barcodes serve to help quantify RNA transcripts and to associate each of the sequences with their cell of origin. In the process of the instant disclosure, however, co-encapsulation of cells with beads for attachment of identifying sequences, is advantageously not required.

### Multi-gRNA Constructs

While the processes of the instant disclosure are currently exemplified using cells having individual guide RNAs integrated and expressed within each cell, it is further contemplated that linking of more than one guide RNA (gRNA) can be performed, for perturbation and transcriptional assessment of individual cells that are multi-gRNA-expressing. Where multiple guides are linked, it is contemplated that a single expressed barcode within a construct that links and expresses, e.g., 2, 3, 4, 5 or more gRNAs (or other exogenous regulatory nucleic acids), can serve as a proxy for presence and expression of the, e.g., 2, 3, 4, 5 or more gRNAs, in such cells as the barcode can be fused with specifically monitored transcripts within each cell, via overlap extension. Pairing of proxy barcodes with gRNA groups can therefore take place with gRNAs. In such embodiments, gRNA sequence identifiers can be barcodes, which provide compressed information regarding vector gRNA contents, and such barcodes can be as short as, e.g., 10-20 nucleotides in length. In embodiments, pairings of proxy barcodes with their cognate gRNA groups can be separately sequenced prior to cellular introduction. Such pre-sequencing of proxy barcodes enables random pooled assembly with subsequent proxy/gRNA group identification. In certain embodiments, the instant disclosure employs overlap extension to provide barcodes as proxy for multiple genes in a gRNA plasmid, with such barcodes then fused to one or more target transcripts during in-droplet amplification processes.

Thus, the synthetic information-bearing amplicon that is ultimately sequenced in massively parallel fashion in the methods of the instant disclosure will tend to include both gRNA information (including multi-gRNA information, e.g., in the form of a barcode) and downstream effect information in the form of panels of surveyed transcripts, which are ultimately resolvable via the in-droplet pairing provided by such overlap extension process (optionally including barcoding) at the single-cell level, even where sequencing is performed in bulk, massively parallel fashion.

### Droplets and Droplet Libraries

In certain embodiments of the instant disclosure, high throughput and high resolution delivery of reagents to individual emulsion droplets is performed, by art-recognized means (refer, e.g., to WO 2016/040476, among others). Emulsion droplets may contain cells, organelles, nucleic acids, proteins, etc., and delivery into droplets is performed through the use of monodisperse aqueous droplets that are generated by a microfluidic device as a water-in-oil emulsion. The droplets are carried in a flowing oil phase and stabilized by a surfactant. In one aspect, single cells or single organelles or single molecules (proteins, RNA, DNA) are encapsulated into uniform droplets from an aqueous solution/dispersion. In a related aspect, multiple cells or multiple molecules may take the place of single cells or single molecules. The aqueous droplets of volume ranging from 1 pL to 10 nL work as individual reactors. Disclosed embodiments provide thousands to tens of thousands or even millions of single cells in droplets which can be processed and analyzed in a single run.

It is contemplated that droplet/emulsion volume depends on both the droplet/emulsion system employed and the size of the input cells. To form cell-incorporating droplets or emulsions, whole cells need to be able to pass through a droplet/emulsion-making microfluidic device without clogging it. Therefore, the droplet/emulsion volume for cell-based microfluidics will tend to be bigger than for DNA-based microfluidics. Thus, in certain embodiments, for encapsulating mammalian cells (e.g., where the diameter of U937 cells is 13 microns), droplet/emulsion volumes of between about 20 pL and about 80 pL are contemplated as optimal. Meanwhile, droplet sizes of as little as about 20 microns might be used for encapsulation of mammalian cells, which would result in droplet volumes of about 4 pL. Thus, mammalian cell droplet sizes of about 4 pL to about 80 pL or more are expressly contemplated.

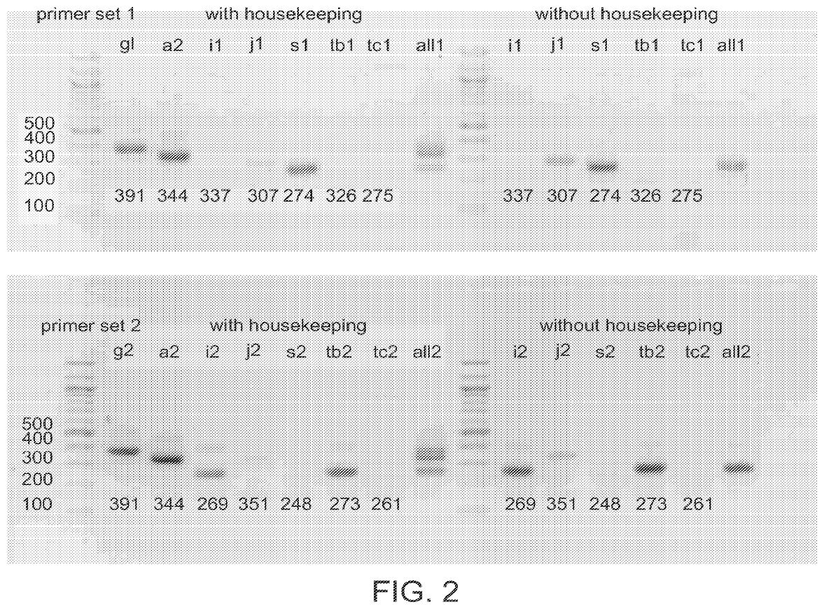

Performance of bulk emulsion are also expressly contemplated. Such bulk emulsions essentially involve combining an oil phase and an aqueous phase together in a tube and shaking/vortexing to form droplets. While such bulk emulsion approaches might be sub-optimal for encapsulating mammalian cells, it is contemplated that bulk emulsion can be a preferred method for encapsulating molecules (i.e. DNA) or microbes. While droplet volumes are polydisperse with a bulk emulsion method, droplet formation using a bulk emulsion process is much easier than using a droplet maker. Exemplary bulk emulsion methods include those of Abil et al. (Nature Protocols 12: 2493-2512—see, e.g., FIG. 2 therein). It is further contemplated that emulsion droplets can also be much smaller, e.g., ranging from about 3 microns to about 25 microns in diameter, depending on the method of emulsifying (sonication vs manual shaking) (refer to Sun et al. Nanoscale Research Letters 12: 434).

With optimization, it is further contemplated that droplet sizes can be made even smaller (e.g., via emulsification methods), particularly for non-mammalian cell applications.

Exemplary microdroplets of the instant disclosure each contain a variety of specific cells, gRNAs (or other regulatory polynucleotides, or polynucleotide-tagged proteins/protein variants) or gRNA-encoding vectors (optionally tagged gRNAs and/or expression-tagged vectors), oligonucleotides, PCR reagents, and optionally molecular barcodes of interest, and synthesis of such loaded microdroplets involves generation and combination of components at preferred conditions, e.g., mixing ratio, concentration, and order of combination.

Methods for producing droplets of a uniform volume at a regular frequency are well known in the art. One method is to generate droplets using hydrodynamic focusing of a dispersed phase fluid and immiscible carrier fluid, such as disclosed in U.S. Publication No. US 2005/0172476 and International Publication No. WO 2004/002627. It is desirable for one of the species introduced at the confluence to be a pre-made library of droplets where the library contains a plurality of reaction conditions (components), e.g., a gRNA library may contain plurality of different gRNAs (or gRNA-encoding vectors) encapsulated as separate library elements for screening their effect on cells, alternatively a library could be composed of a plurality of different primer pairs encapsulated as different library elements for targeted amplification of a collection of loci. The introduction of a library of reaction conditions (reaction components) onto a substrate is achieved by pushing a premade collection of library droplets out of a vial with a drive fluid. The drive fluid is a continuous fluid. The drive fluid may comprise the same substance as the carrier fluid (e.g., a fluorocarbon oil). For example, if a library consists of ten pico-liter droplets is driven into an inlet channel on a microfluidic substrate with a drive fluid at a rate of 10,000 pico-liters per second, then nominally the frequency at which the droplets are expected to enter the confluence point is 1000 per second.

The surfactant and oil combination of microdroplets tends to (1) stabilize droplets against uncontrolled coalescence during the drop forming process and subsequent collection and storage, (2) minimize transport of any droplet contents to the oil phase and/or between droplets, and (3) maintain chemical and biological inertness with contents of each droplet (e.g., no adsorption or reaction of encapsulated contents at the oil-water interface, and no adverse effects on biological or chemical constituents in the droplets). In addition, the surfactant-in-oil solution tends to be coupled with the fluid physics and materials associated with the droplet-forming/filling platform selected. Specifically, oil solutions are selected so as not to swell, dissolve, or degrade the materials used to construct a microfluidic chip, and the physical properties of the oil (e.g., viscosity, boiling point, etc.) are matched to the flow and operating conditions of the selected platform.

A droplet library may be made up of a number of library elements that are pooled together in a single collection (see, e.g., US Patent Publication No. 2010002241). Libraries may vary in complexity from a single library element to 1015 library elements or more. Each library element may be one or more given components at a fixed concentration. The element may be, but is not limited to, cells, organelles, virus, bacteria, yeast, beads, amino acids, proteins, polypeptides, nucleic acids, polynucleotides or small molecule chemical compounds. The element may contain an identifier such as a label. The terms “droplet library” or “droplet libraries” can also be referred to as an “emulsion library” or “emulsion libraries.” These terms are used interchangeably in the art.

A cell library element may include, but is not limited to, T-cells, B-cells, primary cells, cultured cell lines, cancer cells, stem cells, hybridomas, cells obtained from tissue (e.g., retinal or human bone marrow), peripheral blood mononuclear cell, or any other cell type. Cellular library elements are prepared by encapsulating a number of cells from one to hundreds of thousands in individual droplets. The number of cells encapsulated is usually given by Poisson statistics from the number density of cells and volume of the droplet. However, in some cases the number deviates from Poisson statistics as described in Edd et al., “Controlled encapsulation of single-cells into monodisperse picolitre drops.” Lab Chip, 8(8): 1262-1264, 2008. The discrete nature of cells allows for libraries to be prepared in mass with a plurality of cellular variants all present in a single starting media and then that media is broken up into individual droplet capsules that contain at most one cell. These individual droplets capsules are then combined or pooled to form a library consisting of unique library elements. Cell division subsequent to, or in some embodiments following, encapsulation produces a clonal library element.

Examples of cells which are contemplated for use in the instant disclosure include mammalian cells; however the instant disclosure also contemplates methods for profiling host-pathogen cell interactions. To characterize the expression of host-pathogen interactions it can be important to grow the host and pathogen together in the same droplet, without multiple opportunities of pathogen infection.

In embodiments, it is desirable to have exactly one cell per droplet with only a few droplets containing more than one cell (multiplets) when starting with a plurality of cells. In some cases, variations from Poisson statistics may be achieved to provide an enhanced loading of droplets such that there are more droplets with exactly one cell per droplet and few exceptions of empty droplets or droplets containing more than one cell.

Examples of droplet libraries are collections of droplets that have different contents, ranging from cells, beads, nucleic acids, primers, small molecules, proteins, antibodies. Smaller droplets may be in the order of femtoliter (fL) volume drops, which are especially contemplated with droplet dispensors. The volume may range from about 5 to about 600 fL. Larger droplets may range in size from roughly 0.5 micron to 500 micron in diameter, which corresponds to about 1 pico liter to 1 nano liter. However, droplets may be as small as 5 microns and as large as 500 microns. In embodiments, the droplets are at less than 100 microns, about 1 micron to about 100 microns in diameter. In certain embodiments, droplet size is about 20 to 40 microns in diameter (10 to 100 picoliters). Properties of droplet libraries that are optimized during preparation include osmotic pressure balance, uniform size, and size ranges.

In embodiments, the droplets comprised within emulsion libraries may be contained within an immiscible oil which may comprise at least one fluorosurfactant. In some embodiments, the fluorosurfactant comprised within immiscible fluorocarbon oil is a block copolymer consisting of one or more perfluorinated polyether (PFPE) blocks and one or more polyethylene glycol (PEG) blocks. In other embodiments, the fluorosurfactant is a triblock copolymer consisting of a PEG center block covalently bound to two PFPE blocks by amide linking groups. The presence of the fluorosurfactant (similar to uniform size of the droplets in the library) is important for maintaining the stability and integrity of the droplets and is also important for the subsequent use of the droplets within the library for the various biological and chemical assays described herein. The types of fluids (e.g., aqueous fluids, immiscible oils, etc.) and other surfactants that may be utilized in the droplet libraries of the present disclosure have also been described in the art.

Droplet libraries of the instant disclosure may comprise a plurality of aqueous droplets within an immiscible oil (e.g., fluorocarbon oil) which may comprise at least one fluorosurfactant, wherein each droplet is uniform in size and may comprise the same aqueous fluid and may comprise a different library element. Droplet libraries can also be formed by providing a single aqueous fluid which may comprise different library elements, and encapsulating each library element into an aqueous droplet within an immiscible fluorocarbon oil which may comprise at least one fluorosurfactant, where each droplet is uniform in size and may comprise the same aqueous fluid and may comprise a different library element, and pooling the aqueous droplets within an immiscible fluorocarbon oil which may comprise at least one fluorosurfactant, thereby forming the droplet library.

For example, in one type of emulsion library, all different types of elements (e.g., cells or beads), may be pooled in a single source contained in the same medium. After the initial pooling, the cells or beads are then encapsulated in droplets to generate a library of droplets wherein each droplet with a different type of bead or cell is a different library element. The dilution of the initial solution enables the encapsulation process. In some embodiments, the droplets formed will either contain a single cell or will not contain anything, i.e., be empty. In other embodiments, the droplets formed will contain multiple copies of a library element. The cells being encapsulated are generally variants on the same type of cell. In one example, the cells may comprise cancer cells of a tissue biopsy, and each cell type is encapsulated to be screened for cellular transcript-level responsiveness across a panel of gRNAs.

In embodiments, the droplet library may comprise a plurality of aqueous droplets within an immiscible fluorocarbon oil, wherein a single molecule may be encapsulated, such that there is a single molecule contained within a droplet for every 20-60 droplets produced (e.g., 20, 25, 30, 35, 40, 45, 50, 55, 60 droplets, or any integer in between). Single molecules may be encapsulated by diluting the solution containing the molecules to such a low concentration that the encapsulation of single molecules is enabled. In one specific example, a vector encoding for multiple gRNAs and harboring an expressed barcode that identifies the combination of expressed gRNAs harbored thereupon is encapsulated at a very low concentration (e.g., about 20-100 fM) after two hours of incubation such that there is about one copy of the vector per droplet within a population. Formation of such droplet libraries can rely upon limiting dilutions.

Generally, nucleic acid may be extracted from a biological sample by a variety of techniques such as those described by Maniatis, et al., Molecular Cloning: A Laboratory Manual, Cold Spring Harbor, N.Y., pp. 280-281 (1982). Nucleic acid molecules may be single-stranded, double-stranded, or double-stranded with single-stranded regions (for example, stem- and loop-structures).

A biological sample as described herein may be homogenized or fractionated in the presence of a detergent or surfactant. The concentration of the detergent in the buffer may be about 0.05% to about 10.0%. The concentration of the detergent may be up to an amount where the detergent remains soluble in the solution. In one embodiment, the concentration of the detergent is between 0.1% to about 2%. The detergent, particularly a mild one that is nondenaturing, may act to solubilize the sample. Detergents may be ionic or nonionic. Examples of nonionic detergents include triton, such as the Triton™ X series (Triton™ X-100 t-Oct-C6H4-—(OCH2—CH2)xOH, x=9-10, Triton™ X-100R, Triton™ X-114 x=7-8), octyl glucoside, polyoxyethylene(9)dodecyl ether, digitonin, IGEPAL™ CA630 octylphenyl polyethylene glycol, n-octyl-beta-D-glucopyranoside (betaOG), n-dodecyl-beta, Tween™. 20 polyethylene glycol sorbitan monolaurate, Tween™ 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14E06), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant.

Lysis or homogenization solutions may further contain other agents, such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid.

Certain methods of the instant disclosure involve forming sample droplets. In embodiments, the droplets are aqueous droplets that are surrounded by an immiscible carrier fluid. Methods of forming such droplets are shown for example in Link et al. (U.S. patent application numbers 2008/0014589, 2008/0003142, and 2010/0137163), Stone et al. (U.S. Pat. No. 7,708,949 and U.S. patent application number 2010/0172803), Anderson et al. (U.S. Pat. No. 7,041,481 and which reissued as RE41,780) and European publication number EP2047910 to Raindance Technologies Inc. The content of each of which is incorporated by reference herein in its entirety.

In embodiments, the present disclosure also employs systems and methods for manipulating droplets within a high throughput microfluidic system, as have been described in the art.

The sample fluid may typically comprise an aqueous buffer solution, such as ultrapure water (e.g., 18 mega-ohm resistivity, obtained, for example by column chromatography), 10 mM Tris HCl and 1 mM EDTA (TE) buffer, phosphate buffer saline (PBS) or acetate buffer. Any liquid or buffer that is physiologically compatible with nucleic acid molecules can be used. The carrier fluid may include one that is immiscible with the sample fluid. The carrier fluid can be a non-polar solvent, decane (e.g., tetradecane or hexadecane), fluorocarbon oil, silicone oil, an inert oil such as hydrocarbon, or another oil (for example, mineral oil).

In certain embodiments, the carrier fluid may contain one or more additives, such as agents which reduce surface tensions (surfactants). Surfactants can include Tween, Span, fluorosurfactants, and other agents that are soluble in oil relative to water. In some applications, performance is improved by adding a second surfactant to the sample fluid. Surfactants can aid in controlling or optimizing droplet size, flow and uniformity, for example by reducing the shear force needed to extrude or inject droplets into an intersecting channel. This can affect droplet volume and periodicity, or the rate or frequency at which droplets break off into an intersecting channel. Furthermore, the surfactant can serve to stabilize aqueous emulsions in fluorinated oils from coalescing.

In certain embodiments, the droplets may be surrounded by a surfactant which stabilizes the droplets by reducing the surface tension at the aqueous oil interface. Preferred surfactants that may be added to the carrier fluid include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80), and perfluorinated polyethers (e.g., DuPontKrytox 157 FSL, FSM, and/or FSH). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglyceryl esters of natural fatty acids, propylene glycol, sorbitol, polyoxyethylenated sorbitol esters, polyoxyethylene glycol esters, etc.) and alkanolamines (e.g., diethanolamine-fatty acid condensates and isopropanolamine-fatty acid condensates).

In embodiments, a complex tissue or cell line is dissociated into individual cells, which are then encapsulated in droplets together with gRNAs or gRNA-expressing vectors (or cells may be transfected with gRNA-expressing vectors in advance of use), a plurality of oligonucleotide primers and RT-PCR reagents. Each cell is lysed within a droplet; its target transcripts are amplified via RT-PCR, while its expressed gRNAs (or an expressed gRNA identifier sequence) are also amplified via PCR. First, mRNAs are reverse-transcribed into cDNAs while expressed gRNAs or expressed gRNA-identifying sequences are also reverse transcribed (alternatively, a gRNA identifying sequence resident upon a gRNA vector that identifies the gRNA vector but that is not itself expressed can be amplified in parallel by PCR, without the need for reverse transcription of this gRNA identifying sequence). Pairs of primers performing respective primary amplifications of (1) gRNAs or gRNA-identifying sequences and (2) target transcripts (cDNAs) are designed with overlapping or complementary 5′ tail regions of at least one end of each pair of primers, with such overlapping or complementary 5′ tail regions of sufficient length to induce splicing by overlap extension to occur between gRNA sequences or gRNA-indicating sequences at one end of ultimate PCR amplicons and target transcript amplicons (either where each target transcript is independently fused to an associated gRNA or gRNA identifier sequence or optionally where target transcript amplicons are themselves combined in series with one another via splicing by overlap extension between target transcripts, which are then fused to an associated gRNA or gRNA identifier sequence amplicon by the overlap extension process).

In certain embodiments, oligonucleotide tags can be employed, e.g., to tag guide RNAs, guide-associated nucleic acids (e.g., expressed barcodes can serve as an easily identified proxy for identification of the presence of a guide RNA-expressing vector (optionally, a vector that expresses two or more, three or more, four or more, five or more, etc. distinct gRNAs) in a cell or solution), or to tag other cellular transcripts. Such oligonucleotide tags may be detectable by virtue of their nucleotide sequence, or by virtue of a non-nucleic acid detectable moiety that is attached to the oligonucleotide such as but not limited to a fluorophore, or by virtue of a combination of their nucleotide sequence and the nonnucleic acid detectable moiety.

In some embodiments, a detectable oligonucleotide tag may comprise one or more non-oligonucleotide detectable moieties. Examples of detectable moieties may include, but are not limited to, fluorophores, microparticles including quantum dots (Empodocles, et al., Nature 399:126-130, 1999), gold nanoparticles (Reichert et al., Anal. Chem. 72:6025-6029, 2000), microbeads (Lacoste et al., Proc. Natl. Acad. Sci. USA 97(17):9461-9466, 2000), biotin, DNP (dinitrophenyl), fucose, digoxigenin, haptens, and other detectable moieties known to those skilled in the art. In some embodiments, the detectable moieties may be quantum dots. Methods for detecting such moieties are known in the art.

Thus, detectable oligonucleotide tags may be, but are not limited to, oligonucleotides which may comprise unique nucleotide sequences, oligonucleotides which may comprise detectable moieties, and oligonucleotides which may comprise both unique nucleotide sequences and detectable moieties.

In certain embodiments, the droplets are broken by addition of a fluorosurfactant (like perfluorooctanol), washed, and collected. As exemplified herein, pooling of fused amplicons and sequencing can then be performed as described elsewhere herein. In embodiments, paired-end sequences are then computationally resolved to determine which target mRNAs were associated with which gRNAs. In this way, through a single sequencing run, hundreds of thousands (or more) of gRNA-mediated modulations of target transcripts can be simultaneously obtained.

### Microwell Stitch-Seq

In certain embodiments of the instant disclosure, a microwell array such as those known in the art (e.g., a Seq-well array of Gierahn et al. Nature Methods. 14: 395-398) can be employed for sequestration of cells. In an exemplary embodiment, instead of employing the droplet emulsion method described elsewhere herein to compartmentalize input cells, such cells can be loaded into microwell arrays and combined with PCR mix, in a manner that predominantly results in one cell per microwell. Such loaded microwell array can then be sealed with a PCR plate seal and thermocycled in the same manner as described elsewhere herein for droplet-encapsulated cells. Amplification product can then be recovered from the array and subjected to the remainder of the Stitch-seq protocol of the instant disclosure.

### Amplification in Droplets or Microwell Arrays

In an advantageous embodiment, polymerase chain reactions (PCR) are contemplated (see, e.g., US Patent Publication No. 20120219947). Methods of the disclosure may be used for merging sample fluids for conducting any type of chemical reaction or any type of biological assay. In certain embodiments, methods of the invention are used for merging sample fluids for conducting an amplification reaction in a droplet and/or a microwell array. Amplification refers to production of additional copies of a nucleic acid sequence and is generally carried out using polymerase chain reaction or other technologies well known in the art (e.g., Dieffenbach and Dveksler, PCR Primer, a Laboratory Manual, Cold Spring Harbor Press, Plainview, N.Y. [1995]). The amplification reaction may be any amplification reaction known in the art that amplifies nucleic acid molecules, such as polymerase chain reaction, nested polymerase chain reaction, polymerase chain reaction-single strand conformation polymorphism, ligase chain reaction (Barany F. (1991) PNAS 88:189-193; Barany F. (1991) PCR Methods and Applications 1:5-16), ligase detection reaction (Barany F. (1991) PNAS 88:189-193), strand displacement amplification and restriction fragments length polymorphism, transcription based amplification system, nucleic acid sequence-based amplification, rolling circle amplification, and hyper-branched rolling circle amplification.

In certain embodiments, the amplification reaction is the polymerase chain reaction. Polymerase chain reaction (PCR) refers to methods by K. B. Mullis (U.S. Pat. Nos. 4,683,195 and 4,683,202, hereby incorporated by reference) for increasing concentration of a segment of a target sequence in a mixture of genomic DNA without cloning or purification. The process for amplifying the target sequence includes introducing an excess of oligonucleotide primers to a DNA mixture containing a desired target sequence, followed by a precise sequence of thermal cycling in the presence of a DNA polymerase. The primers are complementary to their respective strands of the double stranded target sequence.

To effect amplification, primers are annealed to their complementary sequence within the target molecule. Following annealing, the primers are extended with a polymerase so as to form a new pair of complementary strands. The steps of denaturation, primer annealing and polymerase extension may be repeated many times (i.e., denaturation, annealing and extension constitute one cycle; there may be numerous cycles) to obtain a high concentration of an amplified segment of a desired target sequence. The length of the amplified segment of the desired target sequence is determined by relative positions of the primers with respect to each other, and therefore, this length is a controllable parameter.

Methods for performing PCR in droplets are shown for example in Link et al. (U.S. Patent application numbers 2008/0014589, 2008/0003142, and 2010/0137163), Anderson et al. (U.S. Pat. No. 7,041,481 and which reissued as RE41,780) and European publication number EP2047910 to Raindance Technologies Inc. The content of each of which is incorporated by reference herein in its entirety. Sample fluids and reagents for performing PCR generally include Taq polymerase, deoxynucleotides of type A, C, G and T, magnesium chloride, and forward and reverse primers, all suspended within an aqueous buffer.

Primers may be prepared by a variety of methods including but not limited to cloning of appropriate sequences and direct chemical synthesis using methods well known in the art (Narang et al., Methods Enzymol., 68:90 (1979); Brown et al., Methods Enzymol., 68:109 (1979)). Primers may also be obtained from commercial sources such as Operon Technologies, Amersham Pharmacia Biotech, Sigma, and Life Technologies. The primers may have an identical melting temperature. The lengths of the primers may be extended or shortened at the 5′ end or the 3′ end to produce primers with desired melting temperatures. Also, the annealing position of each primer pair may be designed such that the sequence and length of the primer pairs yield the desired melting temperature. The simplest equation for determining the melting temperature of primers smaller than 25 base pairs is the Wallace Rule (Td=2(A+T)+4(G+C)). Computer programs may also be used to design primers, including but not limited to Array Designer Software (Arrayit Inc.), Oligonucleotide Probe Sequence Design Software for Genetic Analysis (Olympus Optical Co.), NetPrimer, and DNAsis from Hitachi Software Engineering. The TM (melting or annealing temperature) of each primer is calculated using software programs such as Oligo Design, available from Invitrogen Corp.

In droplet embodiments, a droplet containing, e.g., a lysed cell, can be caused to merge with PCR reagents in a second fluid or droplet, thereby producing a droplet that includes Taq polymerase, deoxynucleotides of type A, C, G and T, magnesium chloride, forward and reverse primers, detectably labeled probes, and the target nucleic acid (e.g., target transcripts and/or expressed gRNA(s) of the lysed cell). Once mixed droplets have been produced, the droplets are thermal cycled, resulting in amplification of the target nucleic acid in each droplet. In certain embodiments, the droplets are flowed through a channel in a serpentine path between heating and cooling lines to amplify the nucleic acid in the droplet. The width and depth of the channel may be adjusted to set the residence time at each temperature, which may be controlled to anywhere between less than a second and minutes.

In certain embodiments, the three temperature zones are used for the amplification reaction. The three temperature zones are controlled to result in denaturation of double stranded nucleic acid (high temperature zone), annealing of primers (low temperature zones), and amplification of single stranded nucleic acid to produce double stranded nucleic acids (intermediate temperature zones). The temperatures within these zones fall within ranges well known in the art for conducting PCR reactions. See for example, Sambrook et al. (Molecular Cloning, A Laboratory Manual, 3rd edition, Cold Spring Harbor Laboratory Press, Cold Spring Harbor, N.Y., 2001).

In certain embodiments, the three temperature zones are controlled to have temperatures as follows: 95° C. (TH), 55° C. (TL), 72° C. (TM). The prepared sample droplets flow through the channel at a controlled rate. The sample droplets first pass the initial denaturation zone (TH) before thermal cycling. The initial preheat is an extended zone to ensure that nucleic acids within the sample droplet have denatured successfully before thermal cycling. The requirement for a preheat zone and the length of denaturation time required is dependent on the chemistry being used in the reaction. The samples pass into the high temperature zone, of approximately 95° C., where the sample is first separated into single stranded DNA in a process called denaturation. The sample then flows to the low temperature, of approximately 55° C., where the hybridization process takes place, during which the primers anneal to the complementary sequences of the sample. Finally, as the sample flows through the third medium temperature, of approximately 72° C., the polymerase process occurs when the primers are extended along the single strand of DNA with a thermostable enzyme.

The nucleic acids undergo the same thermal cycling and chemical reaction as the droplets pass through each thermal cycle as they flow through the channel. The total number of cycles in the device is easily altered by an extension of thermal zones. The sample undergoes the same thermal cycling and chemical reaction as it passes through N amplification cycles of the complete thermal device.

In other embodiments, the temperature zones are controlled to achieve two individual temperature zones for a PCR reaction. In certain embodiments, the two temperature zones are controlled to have temperatures as follows: 95° C. (TH) and 60° C. (TL). The sample droplet optionally flows through an initial preheat zone before entering thermal cycling. The preheat zone may be important for some chemistry for activation and also to ensure that double stranded nucleic acid in the droplets is fully denatured before the thermal cycling reaction begins. In an exemplary embodiment, the preheat dwell length results in approximately 10 minutes preheat of the droplets at the higher temperature.

The sample droplet continues into the high temperature zone, of approximately 95° C., where the sample is first separated into single stranded DNA in a process called denaturation. The sample then flows through the device to the low temperature zone, of approximately 60° C., where the hybridization process takes place, during which the primers anneal to the complementary sequences of the sample. Finally, the polymerase process occurs when the primers are extended along the single strand of DNA with a thermostable enzyme. The sample undergoes the same thermal cycling and chemical reaction as it passes through each thermal cycle of the complete device. The total number of cycles in the device is easily altered by an extension of block length and tubing.

In alternative embodiments, it is contemplated herein that non-PCR methods of nucleic acid amplification as are known in the art can be substituted for PCR and/or RT-PCR in certain nucleic acid amplification steps, e.g., at least for one or more rounds of nucleic acid amplification, where such non-PCR methods are employed. Exemplary non-PCR amplification methods include, without limitation, Recombinase Polymerase Amplification (RPA), Rolling Circle Amplification (RCA), and Loop-mediated isothermal amplification (LAMP), among other nucleic acid amplification methods, including isothermal nucleic acid amplification methods. It is contemplated as within the range of expertise of the skilled artisan to design, e.g., appropriately tailed primers to achieve nucleic acid fusions using such non-PCR amplification methods (optionally while also employing PCR amplification methods for certain other rounds of amplification, where non-PCR amplification methods are employed).

After amplification, droplets may be flowed to a detection module for detection of amplification products. The droplets may be individually analyzed and detected using any methods known in the art, such as detecting for the presence or amount of a reporter. Generally, the detection module is in communication with one or more detection apparatuses. The detection apparatuses may be optical or electrical detectors or combinations thereof. Examples of suitable detection apparatuses include optical waveguides, microscopes, diodes, light stimulating devices, (e.g., lasers), photo multiplier tubes, and processors (e.g., computers and software), and combinations thereof, which cooperate to detect a signal representative of a characteristic, marker, or reporter, and to determine and direct the measurement or the sorting action at a sorting module. Further description of detection modules and methods of detecting amplification products in droplets are shown in Link et al. (U.S. patent application numbers 2008/0014589, 2008/0003142, and 2010/0137163) and European publication number EP2047910 to Raindance Technologies Inc.

In exemplified embodiments, droplets are disrupted after amplification of fused amplicons has been performed, and/or arrayed microwells are combined after amplification of fused amplicons has been performed, and amplicons are pooled, cleaned, tagged for sequencing (e.g., via nested amplification and addition of terminal Illumina® adapters), and then sequenced (e.g., using paired-end NGS sequencing).

### Cell Sources

A wide variety of cells, tissues and/or cell lines are envisioned for use as inputs in the methods and compositions of the current disclosure. In certain embodiments, input cells or tissues are obtained from an animal source, including humans, other mammals (e.g., mice, rats, pigs, cats, dogs, and horses), as well as fish, birds, reptiles, insects, mollusks, and other animals. In many embodiments, nucleic acid samples are derived from mammals, particularly primates, especially humans. In some embodiments, nucleic acid samples are derived from livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) nucleic acid samples are from mammals, for example, rodents (e.g., mice, rats, hamsters), rabbits, primates, or swine such as inbred pigs and the like. In certain embodiments input cells can be obtained from microbes—e.g., bacteria, yeast, other fungi, etc. In some embodiments, input cells can be derived from plants including but not limited to crop plants, in particular, corn, wheat, oat, barley, rye, rice, turfgrass, sorghum, millet, sugarcane, cotton, tobacco, canola, oilseed rape, soybean, vegetables, potatoes, Lemna spp., Nicotiana spp., Arabidopsis, alfalfa, bean, flax, pea, safflower, sorghum, sunflower, tobacco, asparagus, beet, broccoli, cabbage, carrot, cauliflower, celery, cucumber, eggplant, lettuce, onion, oilseed rape, pepper, potato, pumpkin, radish, spinach, squash, tomato, zucchini, almond, apple, apricot, banana, blackberry, blueberry, cacao, cherry, coconut, cranberry, date, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut and watermelon.

For application of the compositions and methods of the instant disclosure to input cells that are plant cells, in certain embodiments, suspension plant cells are a specifically contemplated form of input plant cell (though it is also contemplated that the methods of the instant disclosure can also be performed upon adherent plant cells as well), with additional steps as described and well-known in the art likely required to lyse such input plant cells due to their cell wall. The processes disclosed herein otherwise remain the same for input plant cells—i.e., microwell array plant cells with PCR mix and/or encapsulate plant cells with PCR mix in oil droplets, lyse plant cells in the droplets or array components (e.g., within microwells), and then perform the Stitch-seq process as described herein.

In an exemplified embodiment, U937 cells are used. U937 cells are a model cell line originally isolated from histiocytic lymphoma (Sundström C. Int. J. Cancer. 17: 565-77), and are used to study the behavior and differentiation of monocytes. U937 cells mature and differentiate in response to a number of soluble stimuli, adopting the morphology and characteristics of mature macrophages. U937 cells are of the myeloid lineage and so secrete a large number of cytokines and chemokines either constitutively (e.g., IL-1 and GM-CSF) or in response to soluble stimuli. TNFα and recombinant GM-CSF independently promote IL-10 production in U937 cells (Lehmann M H. Mol. Immunol. 35: 479-485).

### CRISPR Systems

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (me) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the RNA. Specifically, the target strand not complementary to crRNA is first cut endonucleolytically, then trimmed 3′→5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821(2012), the entire contents of which is herein incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an Ml strain of Streptococcus pyogenes.” Ferretti et al. Proc. Natl. Acad. Sci. U.S.A. 98:4658-4663(2001); “CRISPR RNA maturation by trans-encoded small RNA and host factor RNase III.” Deltcheva E., et al. Nature 471:602-607(2011); and “A programmable dual-RNA-guided DNA endonuclease in adaptive bacterial immunity.” Jinek M. et al. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:(5): 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al. Science. 337:816-821(2012); Qi et al. “Repurposing CRISPR as an RNA-Guided Platform for Sequence-Specific Control of Gene Expression” (2013) Cell. 28; 152(5): 1173-83, the entire contents of each of which are incorporated herein by reference). For example, the DNA cleavage domain of Cas9 is known to include two subdomains, the HNH nuclease subdomain and the RuvCl subdomain. The HNH subdomain cleaves the strand complementary to the gRNA, whereas the RuvCl subdomain cleaves the noncomplementary strand. Mutations within these subdomains can silence the nuclease activity of Cas9. For example, the mutations D10A and H840A completely inactivate the nuclease activity of S. pyogenes Cas9 (Jinek et al. Science. 337:816-821(2012); Qi et al. Cell. 28; 152(5): 1173-83 (2013)). In some embodiments, proteins comprising fragments of Cas9 are employed. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof.

In some embodiments, cells of the disclosure can include a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In some embodiments, the unmodified CRISPR enzyme has DNA cleavage activity, such as Cas9. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In certain embodiments, a cell of the disclosure expresses a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. As a further example, two or more catalytic domains of Cas9 (RuvC I, RuvC II, and RuvC III or the HNH domain) may be mutated to produce a mutated Cas9 substantially lacking all DNA cleavage activity. In certain preferred embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. Where the enzyme is not SpCas9, mutations may be made at any or all residues corresponding to positions 10, 762, 840, 854, 863 and/or 986 of SpCas9 (which may be ascertained for instance by standard sequence comparison tools. In particular, any or all of the following mutations are preferred in SpCas9: D10A, E762A, H840A, N854A, N863A and/or D986A; as well as conservative substitution for any of the replacement amino acids is also envisaged. The same (or conservative substitutions of these mutations) at corresponding positions in other Cas9s are also preferred. Particularly preferred are D10 and H840 in SpCas9. However, in other Cas9s, residues corresponding to SpCas9 D10 and H840 are also preferred.

An aspartate-to-alanine substitution (D10A) in the RuvC I catalytic domain of SpCas9 was previously engineered to convert the nuclease into a nickase (SpCas9n) (see e.g., Sapranauskas et al., 2011, Nucleic Acis Research, 39: 9275; Gasiunas et al., 2012, Proc. Natl. Acad. Sci. USA, 109:E2579), such that nicked genomic DNA undergoes the high-fidelity homology-directed repair (HDR). Surveyor assay confirmed that SpCas9n does not generate indels at the EMX1 protospacer target. Co-expression of EMX1-targeting chimeric crRNA (having the tracrRNA component as well) with SpCas9 produced indels in the target site, whereas co-expression with SpCas9n did not (n=3). Moreover, sequencing of 327 amplicons did not detect any indels induced by SpCas9n. The same locus was selected to test CRISPR-mediated HR by co-transfecting HEK 293FT cells with the chimeric RNA targeting EMX1, hSpCas9 or hSpCas9n, as well as a HR template to introduce a pair of restriction sites (HindIII and NheI) near the protospacer.

Preferred orthologs have been described in the art. A Cas enzyme may be identified Cas9 as this can refer to the general class of enzymes that share homology to the biggest nuclease with multiple nuclease domains from the type II CRISPR system. Most preferably, the Cas9 enzyme is from, or is derived from, spCas9 or saCas9. By derived, Applicants mean that the derived enzyme is largely based, in the sense of having a high degree of sequence homology with, a wildtype enzyme, but that it has been mutated (modified) in some way as described herein.

It will be appreciated that the terms Cas and CRISPR enzyme are generally used herein interchangeably, unless otherwise apparent. As mentioned above, many of the residue numberings used herein refer to the Cas9 enzyme from the type II CRISPR locus in Streptococcus pyogenes. However, it will be appreciated that this disclosure also contemplates many more Cas9s from other species of microbes, such as SpCas9, SaCa9, St1Cas9 and so forth.

### Splicing by Overlap Extension (SOE)

In certain aspects of the instant disclosure, splicing of nucleic acid sequences is performed using an overlap extension process. Splicing by overlap extension (“SOE”), alternatively referred to as overlap extension polymerase chain reaction (OE-PCR) is performed as known in the art and as disclosed in, e.g., U.S. Pat. No. 5,023,171. The OE-PCR process joins two nucleic acid molecules by first amplifying them by means of polymerase chain reactions (PCR) carried out on each molecule using oligonucleotide primers designed so that the ends of the resultant PCR products contain complementary sequences. When the two PCR products mix, denature and reanneal, the single-stranded DNA strands having the complementary sequences at their 3′ ends anneal and then act as primers for each other. Extension of the annealed area by DNA polymerase produces a double-stranded DNA molecule in which the original molecules are spliced together. Elegantly, the precise sites of nucleic acid splicing can be dictated via primer design. In embodiments of the instant disclosure, OE-PCR is used to fuse initially separate nucleic acid sequences of the following Groups I and II, thereby generating a Group I-Group II fusion nucleic acid that can then be sequenced (optionally tagmented and sequenced) in bulk, with associations between Group I nucleic acids and Group II nucleic acids retained in final sequence products and reflecting original associations that occurred at the single cell/individual droplet level.

Group I Nucleic Acids: guide RNAs (gRNAs) or gRNA identifiers (e.g., a unique identifying sequence/expressed barcode that indicates expression of one or a plurality of gRNAs harbored upon a single vector).
Group II Nucleic Acids: selected target transcripts or fragments thereof (e.g., selected transcripts indicative of gRNA-mediated modulation of cellular pathways).

It is noted that overlap extension has recently been described for fusing native pairs of T-cell receptors and B-cell receptors that are co-expressed in single cells (Tanno et al. Science Advances (2020). doi: 10.1126/sciadv.aay9093; U.S. Patent Publication No. 2020/0216840), allowing for enhanced processing, detection and analysis of such pairings/associations.

### Sequencing Methods

Next-Generation Sequencing (NGS) Approaches

In some aspects, the methods of the instant disclosure employ next-generation sequencing (NGS) approaches. NGS, as defined above, has dominated the DNA sequencing space since its development. It has dramatically reduced the cost of DNA sequencing by enabling a massively-paralleled approach capable of producing large numbers of reads at exceptionally high coverages throughout the genome (Treangen and Salzberg. Nature Reviews Genetics 13: 36-46).

NGS works by first amplifying the DNA molecule and then conducting sequencing by synthesis. The collective fluorescent signal resulting from synthesizing a large number of amplified identical DNA strands allows the inference of nucleotide identity. However, due to random errors, DNA synthesis between the amplified DNA strands would become progressively out-of-sync. Quickly, the signal quality deteriorates as the read-length grows. In order to preserve read quality, long DNA molecules must be broken up into small segments, resulting in a critical limitation of NGS technologies (Treangen and Salzberg). Computational efforts aimed to overcome this challenge often rely on approximative heuristics that may not result in accurate assemblies.

It is noted that long-read sequencing (LRS) technologies offer improvements in the characterization of genetic variation and regions that are difficult to assess with prevailing NGS approaches. Long-Read Sequencing (LRS) is a class of DNA sequencing methods currently under active development (Bleidorn, Christoph. Systematics and Biodiversity 14: 1-8). Long-read sequencing works by reading the nucleotide sequences at the single molecule level, in contrast to existing methods that require breaking long strands of DNA into small segments then inferring nucleotide sequences by amplification and synthesis (“Illumina sequencing technology” PDF). By enabling direct sequencing of single DNA molecules, long-read sequencing (LRS) technologies have the capability to produce substantially longer reads than second generation sequencing (Bleidorn). Such an advantage has critical implications for both genome science and the study of biology in general. However, long-read sequencing data have exhibited much higher error rates than previous technologies, which can complicate downstream genome assembly and analysis of the resulting data (Gupta. Trends in Biotechnology 26: 602-611). These technologies are undergoing active development and it is expected that there will be improvements to the high error rates. For applications that are more tolerant to error rates, such as structural variant calling, long-read sequencing has been found to outperform existing methods. As noted above, however, to date, the throughput obtained using true LRS approaches has also been less than for standard NGS approaches. Thus, in currently preferred embodiments standard NGS approaches are used to identify paired/associated target transcript- and gRNA/gRNA identifier sequence-containing ends of fused amplicons.

Paired-End Sequencing

Certain aspects of the instant disclosure employ NGS methods to obtain associated pairs of target transcript sequences and gRNA sequences (or gRNA identifier sequences) within a sequenced population (e.g., a population of fused amplicons). Such pairing (via overlap extension-mediated fusion) of gRNA (or gRNA identifier) sequences and target transcript sequences therefore allows gRNA-mediated transcriptional changes to be identified at the level of single cells or single droplets. It is expressly contemplated that paired-end sequencing can be performed upon nucleic acid populations of the instant disclosure to obtain such pairs of gRNAs and target transcripts. Paired-end sequencing is known in the art, with exemplary description found in, e.g., Fullwood et al., “Next-generation DNA sequencing of paired-end tags (PET) for transcriptome and genome analyses” Genome Res. 19:521-532 (2009), US 2014/0031241, EP Patent No. 2,084,295 and U.S. Pat. No. 7,601,499.

### T-Cell Activation

In certain embodiments, transcriptional profiles associated with T-cell differentiation and/or activation are assessed. A T cell is a type of lymphocyte. The T cell is originated from hematopoietic stem cells (Hematopoietic Stem Cells—stemcells.nih.gov), which are found in the bone marrow; however, the T cell matures in the thymus gland (hence the name) and plays a central role in the immune response. T cells can be distinguished from other lymphocytes by the presence of a T-cell receptor on the cell surface. These immune cells originate as precursor cells, derived from bone marrow (Alberts et al. Molecular Biology of the Cell. Garland Science: New York, NY pg 1367), and develop into several distinct types of T cells once they have migrated to the thymus gland. T cell differentiation continues even after they have left the thymus.

Groups of specific, differentiated T cells have an important role in controlling and shaping the immune response by providing a variety of immune-related functions. One of these functions is immune-mediated cell death, and it is carried out by T cells in several ways: CD8+ T cells, also known as “killer cells”, are cytotoxic—this means that they are able to directly kill virus-infected cells as well as cancer cells. CD8+ T cells are also able to utilize small signalling proteins, known as cytokines, to recruit other cells when mounting an immune response. A different population of T cells, the CD4+ T cells, function as “helper cells”. Unlike CD8+ killer T cells, these CD4+ helper T cells function by indirectly killing cells identified as foreign: they determine if and how other parts of the immune system respond to a specific, perceived threat. Helper T cells also use cytokine signalling to influence regulatory B cells directly, and other cell populations indirectly. Regulatory T cells are yet another distinct population of these cells that provide the critical mechanism of tolerance, whereby immune cells are able to distinguish invading cells from “self”—thus preventing immune cells from inappropriately mounting a response against oneself (which would by definition be an “autoimmune” response). For this reason these regulatory T cells have also been called “suppressor” T cells. These same self-tolerant cells are co-opted by cancer cells to prevent the recognition of, and an immune response against, tumor cells.

T cells are grouped into a series of subsets based on their function. CD4 and CD8 T cells are selected in the thymus, but undergo further differentiation in the periphery to specialized cells which have different functions. T cell subsets were initially defined by function, but also have associated gene or protein expression patterns.

Antigen-naïve T cells expand and differentiate into memory and effector T cells after they encounter their cognate antigen within the context of an MHC molecule on the surface of a professional antigen presenting cell (e.g., a dendritic cell). Appropriate co-stimulation must be present at the time of antigen encounter for this process to occur. Historically, memory T cells were thought to belong to either the effector or central memory subtypes, each with their own distinguishing set of cell surface markers (Sallusto et al. Nature. 401 (6754): 708-712). Subsequently, numerous new populations of memory T cells were discovered including tissue-resident memory T (Trm) cells, stem memory TSCM cells, and virtual memory T cells. The single unifying theme for all memory T cell subtypes is that they are long-lived and can quickly expand to large numbers of effector T cells upon re-exposure to their cognate antigen. By this mechanism they provide the immune system with “memory” against previously encountered pathogens. Memory T cells may be either CD4+ or CD8+ and usually express CD45RO (Akbar et al. J. Immunol. 140 (7): 2171-8).

Memory T cell subtypes include:
Central memory T cells (TCM cells) express CD45RO, C-C chemokine receptor type 7 (CCR7), and L-selectin (CD62L). Central memory T cells also have intermediate to high expression of CD44. This memory subpopulation is commonly found in the lymph nodes and in the peripheral circulation. (Note: CD44 expression is usually used to distinguish murine naïve from memory T cells).
Effector memory T cells (TEM cells and TEMRA cells) express CD45RO but lack expression of CCR7 and L-selectin. They also have intermediate to high expression of CD44. These memory T cells lack lymph node-homing receptors and are thus found in the peripheral circulation and tissues (Willinger et al. Journal of Immunology. 175 (9): 5895-903). TEMRA stands for terminally differentiated effector memory cells re-expressing CD45RA, which is a marker usually found on naïve T cells (Koch et al. Immunity & Ageing. 5 (6): 6).
Tissue resident memory T cells (TRM) occupy tissues (skin, lung, etc.) without recirculating. One cell surface marker that has been associated with TRM is the intern αeβ7, also known as CD103 (Shin and Iwasaki. Immunological Reviews. 255 (1): 165-81).
Virtual memory T cells differ from the other memory subsets in that they do not originate following a strong clonal expansion event. Thus, although this population as a whole is abundant within the peripheral circulation, individual virtual memory T cell clones reside at relatively low frequencies. One theory is that homeostatic proliferation gives rise to this T cell population. Although CD8 virtual memory T cells were the first to be described (Lee et al. Trends in Immunology. 32 (2): 50-56), it is now known that CD4 virtual memory cells also exist.

Activation of CD4+ T cells occurs through the simultaneous engagement of the T-cell receptor and a co-stimulatory molecule (like CD28, or ICOS) on the T cell by the major histocompatibility complex (MHCII) peptide and co-stimulatory molecules on the APC. Both are required for production of an effective immune response; in the absence of co-stimulation, T cell receptor signalling alone results in anergy. The signalling pathways downstream from co-stimulatory molecules usually engages the PI3K pathway generating PIP3 at the plasma membrane and recruiting PH domain containing signaling molecules like PDK1 that are essential for the activation of PKC-0, and eventual IL-2 production. Optimal CD8+ T cell response relies on CD4+ signaling (Williams and Bevan. Annual Review of Immunology. 25 (1): 171-92). CD4+ cells are useful in the initial antigenic activation of naïve CD8 T cells, and sustaining memory CD8+ T cells in the aftermath of an acute infection. Therefore, activation of CD4+ T cells can be beneficial to the action of CD8+ T cells (Janssen et al. Nature. 421 (6925): 852-6; Shedlock and Shen. Science. 300 (5617): 337-9; Sun et al. Nature Immunology. 5 (9): 927-33).

The first signal is provided by binding of the T cell receptor to its cognate peptide presented on MHCII on an APC. MHCII is restricted to so-called professional antigen-presenting cells, like dendritic cells, B cells, and macrophages, to name a few. The peptides presented to CD8+ T cells by MHC class I molecules are 8-13 amino acids in length; the peptides presented to CD4+ cells by MHC class II molecules are longer, usually 12-25 amino acids in length (Rolland and O'Hehir, “Turning off the T cells: Peptides for treatment of allergic Diseases,” Today's life science publishing, 1999, Page 32), as the ends of the binding cleft of the MHC class II molecule are open.

The second signal comes from co-stimulation, in which surface receptors on the APC are induced by a relatively small number of stimuli, usually products of pathogens, but sometimes breakdown products of cells, such as necrotic-bodies or heat shock proteins. The only co-stimulatory receptor expressed constitutively by naïve T cells is CD28, so co-stimulation for these cells comes from the CD80 and CD86 proteins, which together constitute the B7 protein, (B7.1 and B7.2, respectively) on the APC. Other receptors are expressed upon activation of the T cell, such as OX40 and ICOS, but these largely depend upon CD28 for their expression. The second signal licenses the T cell to respond to an antigen. Without it, the T cell becomes anergic, and it becomes more difficult for it to activate in future. This mechanism prevents inappropriate responses to self, as self-peptides will not usually be presented with suitable co-stimulation. Once a T cell has been appropriately activated (i.e. has received signal one and signal two) it alters its cell surface expression of a variety of proteins. Markers of T cell activation include CD69, CD71 and CD25 (also a marker for Treg cells), and HLA-DR (a marker of human T cell activation). CTLA-4 expression is also up-regulated on activated T cells, which in turn outcompetes CD28 for binding to the B7 proteins. This is a checkpoint mechanism to prevent over activation of the T cell. Activated T cells also change their cell surface glycosylation profile (Maverakis et al. J Autoimmun. 57 (6): 1-13).

While in most cases activation is dependent on TCR recognition of antigen, alternative pathways for activation have been described. For example, cytotoxic T cells have been shown to become activated when targeted by other CD8 T cells leading to tolerization of the latter (Milstein et al. Blood. 117 (3): 1042-52).

A unique feature of T cells is their ability to discriminate between healthy and abnormal (e.g., infected or cancerous) cells in the body (Feinerman et al. Mol. Immunol. 45 (3): 619-31). Healthy cells typically express a large number of self derived pMHC on their cell surface and although the T cell antigen receptor can interact with at least a subset of these self pMHC, the T cell generally ignores these healthy cells. However, when these very same cells contain even minute quantities of pathogen derived pMHC, T cells are able to become activated and initiate immune responses. The ability of T cells to ignore healthy cells but respond when these same cells contain pathogen (or cancer) derived pMHC is known as antigen discrimination.

T cell exhaustion is a state of dysfunctional T cells. It is characterized by progressive loss of function, changes in transcriptional profiles and sustained expression of inhibitory receptors. At first cells lose their ability to produce IL-2 and TNFα followed by the loss of high proliferative capacity and cytotoxic potential, eventually leading to their deletion. Exhausted T cells typically indicate higher levels of CD43, CD69 and inhibitory receptors combined with lower expression of CD62L and CD127. Exhaustion can develop during chronic infections, sepsis and cancer (Yi et al. Immunology. 129 (4): 474-81). Exhausted T cells preserve their functional exhaustion even after repeated antigen exposure (Wang et al. Front Immunol. 9: 219).

### Kits

All or some of the essential materials and reagents required for carrying out methods of the disclosure may be provided in a kit. The kit may comprise one or more of oligonucleotide primers, vectors (including gRNA expression vectors and/or Cas enzyme expression vectors), enzymes (including, e.g., reverse transcriptase, polymerase, etc., and/or enzyme-encoding nucleic acids), sequencing reagents, buffers, ribonucleotides, deoxyribonucleotides, salts, and so forth corresponding to at least some embodiments of the provided methods. Embodiments of kits may comprise reagents for the detection and/or use of a control cell, sample, nucleic acid or enzyme, for example. Kits may provide instructions, controls, reagents, containers, and/or other materials for performing various assays or other methods (e.g., those described herein) using the enzymes of the disclosure.

The kits generally may comprise, in suitable means, distinct containers for each individual reagent, primer, and/or enzyme. In specific embodiments, the kit further comprises instructions for producing, testing, and/or using components of the disclosure. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein. The instant disclosure also provides kits containing agents of this disclosure for use in the methods of the present disclosure. Kits of the instant disclosure may include one or more containers comprising an agent and/or composition of this disclosure. Suitable packaging includes, but is not limited to, vials, bottles, jars, flexible packaging (e.g., sealed Mylar or plastic bags), and the like. The container may further comprise a pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Stitch-Seq Enables High-Throughput Assessment of Guide RNA-Mediated Transcriptome Perturbations of Droplet-Encapsulated Single T-Cells

In the current example, gRNA library-mediated perturbations are performed upon a population of naïve T-cells (CROP-seq), and the expression of genes related to T-cell differentiation is analyzed, to quickly determine the effect of each gRNA-mediated perturbation on differentiation.

To perform the process, a CROP-seq library of naïve T-cells engineered to express Cas9 and individual gRNAs of a gRNA library is encapsulated within individual water-in-oil droplets together with RT-PCR reagents, paired oligonucleotide primers for amplification of a panel of target transcripts relevant to assessment of T-cell differentiation state and paired oligonucleotide primers for amplification of expressed gRNAs. gRNAs are integrated into individual T-cell genomes using lentivirus, with the gRNA expression construct located in the delta U3 region of the lentiviral vector, which allows for cellular expression from the vector to create a functional gRNA and part of a 3′-UTR. gRNA-expressing cells are initially selected by flow cytometry for those cells that express a gRNA-associated GFP molecule. During the droplet-encapsulation process, T-cells are lysed via treatment with a Betaine solution (4 M, Sigma-Aldrich), which allows for co-encapsulated primers and RT-PCR reagents to access target transcripts and expressed gRNAs.

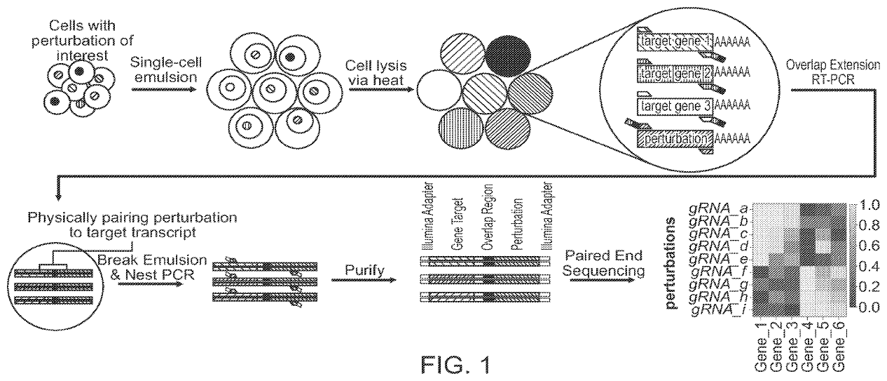

RT-PCR is then performed upon the lysed cells within droplets, and overlap extension is employed during PCR amplification to join target transcript amplicons with copies of associated cell-expressed gRNAs, ultimately forming fused amplicons (FIG. 1). After thereby obtaining fused amplicons within droplets, droplets are burst via addition of a large volume of perfluorooctanol in 6×SSC, thereby releasing a population of fused amplicons. Fused amplicons are then pooled and cleaned in initial preparation for sequencing and subjected to nested amplification to attach Illumina® adapter sequences to fused amplicon regions to be sequenced, in further preparation for paired-end sequencing (FIG. 1). Paired-end NGS sequencing is then performed upon adapter-tagged target transcript-gRNA fusions in bulk, using an Illumina® platform. Resulting sequence data are analyzed to identify target transcript levels and the identities of associated expressed gRNAs, at the individual cell/droplet level, across the population of droplet-encapsulated T-cells. Such analyses reveal specific gRNAs within the population of droplet-encapsulated T-cells that provoke differentiation state changes in individual T-cells of the population of droplet-encapsulated T-cells, including, e.g., identification of gRNAs that promote differentiation of naïve T-cells to memory, activated or exhausted states.

By physically linking the gRNA and the expressed transcripts of interest, the currently disclosed process drastically simplifies the workflow for capturing perturbation effects on gene expression, by circumventing the use of beads (as would need to be employed in the known CROP-seq method). The currently disclosed process thereby enables large increases in screen scale, which provides for quick identification of perturbations that warrant additional in-depth testing (i.e., once gRNA-mediated perturbations are identified that drastically change the expression of genes of interest in individual cells, further exploration of such gRNA effects can then be performed.

### Example 2: Optimization of Oligonucleotide Primers for Multiplex Use in Amplification of Target Transcripts During the Stitch-Seq Process

Initial sets of oligonucleotide primers for stitched (via overlap extension) amplification of target transcripts with gRNA sequences or gRNA identifying sequences were designed and examined for efficacy. Target transcripts of interest for amplification and stitching included IRF3 (“i1” and “i2” in FIG. 2), DNA JC13 (“j1” and “j2” in FIG. 2), STINGI (“s1” and “s2” in FIG. 2), TBK1 (“tb1” and “tb2” in FIG. 2) and TCF7 (“tc1” and “tc2” in FIG. 2). Initial stitching (via overlap extension (OE)) was performed in multiplex, and was followed by individual nested PCR amplifications. Primer optimization was achieved via normal PCR, standard dilution and stitching (via OE). While product levels varied significantly across stitched transcript amplifications examined, respective stitched products were obtained with at least one primer set, for each of IRF3, DNA JC13, STINGI and TBK1, while stitched TCF7 amplicons were observed at only very low levels in primer set 2 (FIG. 2). Housekeeping genes are known to sequester gRNA during stitching, and the impact of removal of housekeeping genes upon formation of stitched (fused) amplicons was also examined, and was identified to improve yields of a number of target transcript amplicons (FIG. 2).

### Example 3: Preliminary Uniform Droplet Populations Prepared for the Stitch-Seq Process

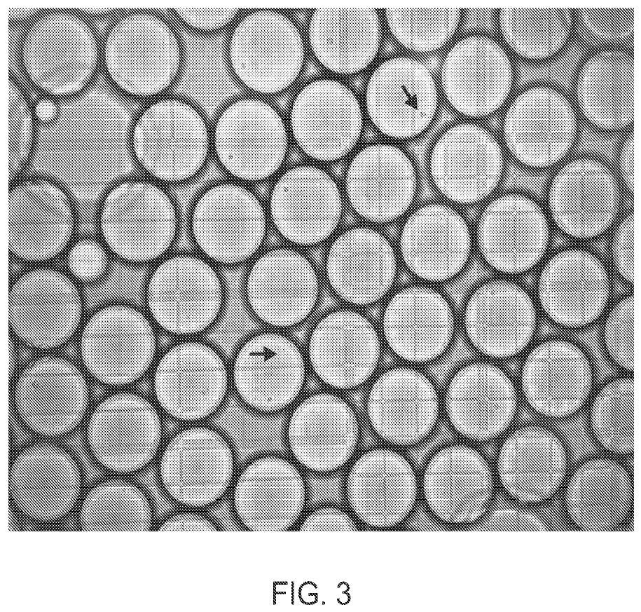

A population of droplets was prepared and examined, using droplet digital PCR, which is a process similar to the known DROP-seq process. Specifically, a dropletizer took in cells and reagents, and resulting droplets were stable enough to undergo PCR amplification in droplets. Prior to droplet formation, cells were contacted with trypsin for two minutes, and cells were not treated with Betaine until immediately before droplet encapsulation occurred. A population of droplets was thereby prepared, and was examined under magnification, which revealed that the population of droplets possessed reasonably uniform size and minimal multiplets (droplets with multiple cells) (FIG. 3). The projected throughput of the current approach using droplet sizes as readily obtained was estimated to be up to about 100,000 cells/mL.

### Example 4: Fidelity and Quantitative Benchmarking of Stitch-Seq

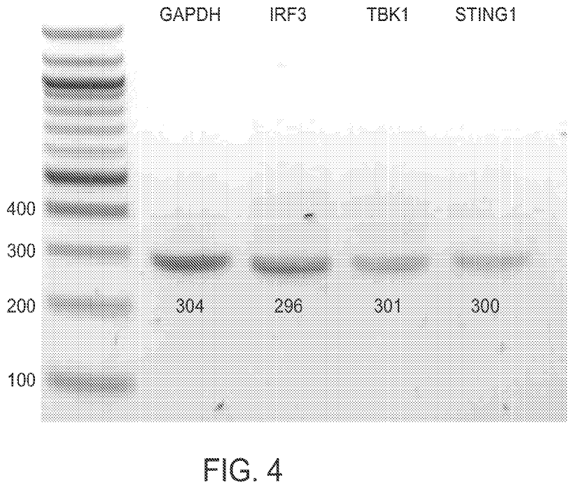

To demonstrate the fidelity of Stitch-seq, multiplex, droplet-based Stitch PCR was performed to fuse cognate gRNAs with a series of gene products, and nested PCR products for gRNA-fused amplicons containing GAPDH, IRF3, TBK1 and STINGI, respectively, were obtained and imaged via gel electrophoresis. Discrete bands were observed for fusion product amplicons of each gene with cognate gRNA (FIG. 4). In such experiments, U937 cells were dropletized at 100k cells/mL.

Quantitative benchmarking of Stitch-seq was performed by obtaining and plotting the percentage of reads (log scale) that aligned to each synthetic target gene (at different initial concentrations of synthetic target gene) for a range of gRNA concentrations, as compared to the expected proportion of reads given the initial concentration. A highly correlated R² value of 0.984 was observed for the 16.2 nM gRNA concentration.

### Example 5: Droplet Stability after PCR, Droplet Mixing and Reaction Fidelity

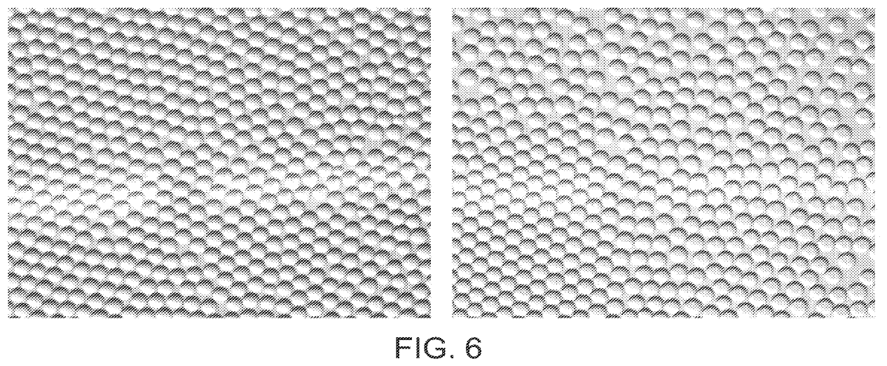

To examine droplet stability during the Stitch-seq process, oil droplets containing complete Stitch-seq reactions were produced and then imaged, both before and after thermocycling for comparison. Stitch-seq droplets were thereby observed to have exhibited robust stability throughout the Stitch-seq process (FIG. 6).

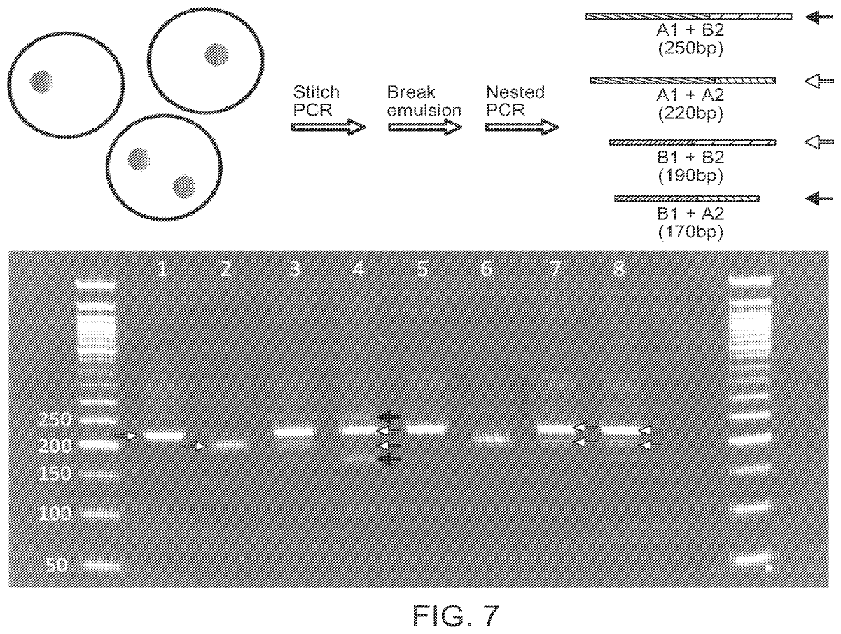

To examine the reaction fidelity of Stitch-seq, two engineered cell lines were subjected to Stitch reactions in bulk cell populations or as dropletized cells, with each of the engineered cell lines employed stably expressing either reporter A or reporter B. Unique parts of the transcripts (A1, A2, B1, B2) were amplified such that A1 or B1 could stitch to A2 or B2, each generating fragments of different sizes depending on what was amplified (A1+A2 and B1+B2 single cell line amplicons, and crossover cell line product amplicons A1+B2 and B1+A2; FIG. 7). Gel electrophoresis was employed to visualize respective, nested PCR products obtained from: (1) a bulk Stitch PCR performed upon cells containing reporter A only (only fusion A1+A2 was possible in such cells, FIG. 7, lane 1); (2) a nested PCR product obtained from a bulk Stitch PCR performed upon cells containing reporter B, where only B1+B2 was possible (FIG. 7, lane 2); (3) the product of a nested PCR performed upon amplicons from the Stitch PCRs of conditions 1 and 2, to identify any crossover during the nested PCR reaction (no crossover was observed; FIG. 7, lane 3); (4) nested PCR product of cells containing reporter A and cells containing reporter B that were input into a bulk Stitch PCR together (all four expected fusion PCR products were observed; FIG. 7, lane 4); (5) nested PCR product obtained from a droplet Stitch PCR performed upon cells containing reporter A (only fusion A1+A2 was possible in such dropletized cells, FIG. 7, lane 5); (6) nested PCR product from a droplet Stitch PCR performed upon cells containing reporter B (only fusion B1+B2 was possible in such dropletized cells, FIG. 7, lane 6); (7) nested PCR product from a droplet Stitch PCR of cells with reporter A dropletized separately from cells with reporter B. However, droplets were mixed for the Stitch PCR, to identify any droplet merging that might have occurred in mixing droplets during the Stitch PCR (only A1+A2 and B1+B2 fusion products were observed, indicating effectively no droplet merging occurred when droplets were mixed; and (8) nested PCR product of a droplet Stitch PCR performed upon cells with reporter A and cells with reporter B dropletized together for the Stitch PCR, to identify the prevalence and impact of doublets produced during dropletization. If there were no doublets formed during dropletization, no cell A/cell B mixing during the Stitch PCR would have occurred, and no cross-product amplification would be detected during the nested PCR, resulting in only two bands (which was observed; FIG. 7, lane 8). Were doublets/mixing/crossover to have occurred during generation and use of dropletized cell populations, there would have been 4 bands observed, as shown in FIG. 7, lane 4. No such four-way outcome of dropletized cell populations was observed (even where droplets were mixed). Such results demonstrated that there was no nested PCR crossover (FIG. 7, lane 3), no droplet merging (FIG. 7, lane 7), and minimal doublets formed (FIG. 7, lane 8) throughout Stitch-seq, meaning that the droplets were successfully compartmentalizing each cell for Stitch PCR, and that Stitch-seq maintained fidelity of the reaction inputs (FIG. 7).

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A method for identifying within a population of individually sequestered or discretely identifiable cells one or more target transcripts and one or more exogenous polynucleotides in an individual cell, the method comprising:
(a) preparing or providing a population of individually sequestered or discretely identifiable cells, wherein a plurality of said cells comprises:
an individual cell harboring one or more exogenous polynucleotides or comprising a nucleic acid vector capable of expressing one or more exogenous polynucleotides;
nucleic acid amplification reagents; and
a plurality of oligonucleotides comprising:
(i) a first pair of oligonucleotide primers for amplifying an exogenous polynucleotide in the individually sequestered or discretely identifiable cell; and
(ii) a second pair of oligonucleotide primers for amplifying a target transcript of the individually sequestered or discretely identifiable cell,
wherein the first pair of oligonucleotide primers possesses a primer having a 5′-terminal region of sequence that is the same or complementary to a 5′-terminal region of sequence of a primer of the second pair of oligonucleotide primers, wherein the 5′-terminal region that is the same or complementary between the first pair of oligonucleotide primers and the second pair of oligonucleotide primers is of sufficient length to allow for amplification-mediated joining of an amplicon of the first pair of oligonucleotide primers and an amplicon of the second pair of oligonucleotide primers into a fused amplicon,
wherein the individually sequestered or discretely identifiable cell is lysed to render contents of the cell accessible in a manner that maintains the sequestering or discrete identification of the lysed cell contents;
(b) performing polymerase-mediated primer extension and optionally thermal cycling upon the population of lysed cell contents under conditions suitable for generating fused amplicons comprising the amplicon of the first pair of oligonucleotide primers and the amplicon of the second pair of oligonucleotide primers by overlap extension, thereby generating fused amplicons within the individually sequestered or discretely identifiable lysed cell contents;
(c) recovering fused amplicons from the population of lysed cell contents; and
(d) obtaining sequence information from the fused amplicons using a sequencing method capable of obtaining sequences from both ends of individual fused amplicon sequences and identifying as a pair said sequences obtained from both ends of the same individual fused amplicon, thereby identifying in the population of individually sequestered or discretely identifiable cells one or more target transcripts and one or more exogenous polynucleotides within the individually sequestered or discretely identifiable cell.

2. The method of claim 1, wherein the individually sequestered or discretely identifiable cells:
are droplet-encapsulated or emulsion-encapsulated;
are present in a hydrogel, optionally wherein the population of individually sequestered or discretely identifiable cells has been split and pool labeled;
are present in a microfluidic chip; or
are present in an array, optionally wherein the population of individually sequestered or discretely identifiable cells is present in a microwell array and/or a plate, optionally wherein the microwell array is a microwell array comprising a sub-nanoliter fluid volume per well and/or the plate is a 96-well or 384-well plate.

3. The method of claim 1, wherein the nucleic acid amplification reagents comprise reagents selected from the group consisting of Polymerase Chain Reaction (PCR) reagents, Recombinase Polymerase Amplification (RPA) reagents, Rolling Circle Amplification (RCA) reagents, Loop-mediated isothermal amplification (LAMP) reagents or other isothermal amplification reagents, optionally wherein the nucleic acid amplification reagents comprise PCR reagents, optionally wherein the nucleic acid amplification reagents comprise reverse transcriptase PCR (RT-PCR) reagents.

4. The method of claim 1, wherein the polymerase-mediated primer extension and optionally thermal cycling performed upon the population of lysed cell contents under conditions suitable for generating fused amplicons comprising the amplicon of the first pair of oligonucleotide primers and the amplicon of the second pair of oligonucleotide primers by overlap extension comprises performing one or more rounds of amplification selected from the group consisting of Polymerase Chain Reaction (PCR), Recombinase Polymerase Amplification (RPA), Rolling Circle Amplification (RCA), Loop-mediated isothermal amplification (LAMP) or other isothermal amplification, upon the population of lysed cell contents, optionally wherein PCR and thermal cycling are performed upon the population of lysed cell contents, optionally wherein reverse transcriptase PCR (RT-PCR) and thermal cycling are performed upon the population of lysed cell contents.

5. The method of claim 1, wherein the population of individually sequestered or discretely identifiable cells harbors or expresses a polynucleotide-guided protein capable of interacting with the one or more exogenous polynucleotides, optionally wherein the polynucleotide-guided protein is a polynucleotide-guided nuclease or a nuclease-dead functional variant thereof, optionally wherein the polynucleotide-guided protein is a Cas enzyme or is RISC, optionally wherein the Cas enzyme is a Cas9 or Cas13a enzyme, optionally wherein the Cas enzyme is dCAS9VPR or dCAS9-KRAB.

6. The method of claim 1, wherein the one or more exogenous polynucleotides is capable of interacting with a polynucleotide-guided protein.

7. The method of claim 1, wherein the one or more exogenous polynucleotides comprise a nucleic acid sequence that identifies expression of one or more exogenous polynucleotides capable of interacting with a polynucleotide-guided protein

8. The method of claim 1, wherein identifying in the population of individually sequestered or discretely identifiable cells one or more target transcripts and one or more exogenous polynucleotides identifies the one or more target transcripts and the one or more exogenous polynucleotides as co-expressed.

9. The method of claim 1, wherein the population of individually sequestered or discretely identifiable cells comprises a nucleic acid vector or nucleic acid insert capable of expressing the one or more exogenous polynucleotides, optionally wherein the population of individually sequestered or discretely identifiable cells expresses the one or more exogenous polynucleotides.

10. The method of claim 1, wherein the one or more exogenous polynucleotides comprise a guide RNA (gRNA), optionally wherein the one or more exogenous polynucleotides are gRNAs.

11. The method of claim 1, further comprising comparing identities and levels of target transcripts and exogenous polynucleotides in the population of individually sequestered or discretely identifiable cells to identify exogenous polynucleotide-mediated gene perturbations in individual cells of the population of cells.

12. The method of claim 1, wherein the population of individually sequestered or discretely identifiable cells is a population of individually sequestered or discretely identifiable cells capable of acting as a cellular factory, optionally wherein the population of individually sequestered or discretely identifiable cells comprises Chinese Hamster Ovary (CHO) cells and/or Human Embryonic Kidney (HEK) cells.

13. The method of claim 1, wherein the population of individually sequestered or discretely identifiable cells is a population of individually sequestered or discretely identifiable mammalian cells, optionally a population of individually sequestered or discretely identifiable mammalian cell line cells, optionally a population of U937 lymphoma cell line cells.

14. The method of claim 1, wherein the population of individually sequestered or discretely identifiable cells is a population of primary cells.

15. The method of claim 1, wherein:
the population of individually sequestered or discretely identifiable cells comprises a population of individually sequestered or discretely identifiable non-mammalian cells, optionally wherein the population of individually sequestered or discretely identifiable cells comprises a population of microbial cells, optionally wherein the population of individually sequestered or discretely identifiable cells comprises a population of plant, bacteria and/or yeast cells, optionally wherein the population of individually sequestered or discretely identifiable plant cells comprises a population of suspension plant cells;
the nucleic acid amplification reagents comprise reverse transcriptase, a DNA polymerase and one or more primers selected from the group consisting of: poly-T-tailed oligonucleotide primers, primers for specific amplification of the one or more exogenous polynucleotides capable of interacting with a polynucleotide-guided protein (or expressed polynucleotide proxy therefor), and primers for targeted transcript of interest amplification, optionally wherein the DNA polymerase comprises a thermostable DNA polymerase that enables PCR, optionally wherein the thermostable DNA polymerase is a Taq DNA polymerase, optionally wherein the Taq DNA polymerase is AmpliTaq;
the first pair of oligonucleotide primers amplifies a gRNA or RNAi agent sequence, optionally a gRNA or RNAi agent sequence of a gRNA or RNAi agent library, optionally wherein the gRNA or RNAi agent library comprises between about 40 and about 500,000 or more gRNAs and/or RNAi agents;
the first pair of oligonucleotide primers amplifies a nucleic acid sequence that identifies expression of a plurality of gRNAs or RNAi agents, optionally wherein the plurality of gRNAs or RNAi agents and the sequence that identifies expression of the plurality of gRNAs or RNAi agents are contained on a single vector, optionally wherein the single vector is a plasmid, optionally wherein the plurality of gRNAs or RNAi agents comprises three or more gRNAs or RNAi agents, optionally four or more gRNAs or RNAi agents, optionally five or more gRNAs or RNAi agents, optionally five to twenty gRNAs or RNAi agents, optionally ten to twenty gRNAs or RNAi agents;
the one or more target transcripts is capable of defining a state selected from the group consisting of a cellular differentiation state, a cellular activation state, a cellular stress response state, and a cellular homeostatic state;
the one or more target transcripts comprise one or more interferon stimulated gene transcripts (ISGs), optionally wherein one or more of the ISGs is selected from the group consisting of ADAR1, ISG15, USP18, STING, MDA5, PKR, EIF2a, ATF4, IRF9, RIG1, TBK1, IRF3 and PD-L1;
the one or more target transcripts comprise one or more target transcripts selected from the group consisting of IRF3, DNA JC13, STINGI, TBK1 and TCF7;
the one or more target transcripts comprises a panel of transcripts for assessment of B-cell activation and differentiation status, optionally wherein the panel of transcripts comprises B-cell receptors (BCRs), optionally wherein B-cells are identified as having a differentiation status selected from the group consisting of naïve, memory, activated and plasmoblast;
the one or more target transcripts comprise a plurality of target transcripts, wherein individual droplets, hydrogel elements, microfluidic chip chambers, or array elements of the plurality of droplets, hydrogel elements, microfluidic chip chambers, or array elements comprise respective pairs of oligonucleotide primers for amplifying each target transcript of the plurality of target transcripts, optionally wherein each of the respective pairs of oligonucleotide primers is designed for fusion by overlap extension of the target transcript amplicon with the amplicon of the first pair of oligonucleotide primers, optionally where fusion of one or more target transcript amplicons with an associated gRNA amplicon occurs via intervening fusions with other target transcript amplicons within the individual droplet, hydrogel element, microfluidic chip chamber, or array element, optionally wherein amplification of the plurality of target transcripts is multiplexed;
the individually sequestered or discretely identifiable cell is lysed by heating or by chemical means, optionally wherein the lysis by heating is performed during performance of nucleic acid amplification;
the individually sequestered or discretely identifiable cell is contacted with a Betaine solution (4 M, Sigma-Aldrich), optionally wherein the individually sequestered or discretely identifiable cell is lysed while a population of droplets is being prepared;
the population of individually sequestered or discretely identifiable cells does not comprise microbeads;
step (c) recovering fused amplicons from the population of individually sequestered or discretely identifiable cells comprises breaking open a population of droplets or emulsions, optionally wherein breaking open the population of droplets or emulsions comprises contacting the population of droplets or emulsions with a reagent that destabilizes the oil-water interface of the droplets, optionally wherein the reagent that destabilizes the oil-water interface is a large volume of high-salt solution, optionally wherein the reagent that destabilizes the oil-water interface is a large volume (e.g., 30 mL) of perfluorooctanol (PFO) in 6×SSC or is a small volume (e.g., 200 μL) of 20% PFO, optionally wherein the small volume of 20% PFO is in HFE-7500 3M™ Novec™ engineered fluid;
step (c) recovering fused amplicons from the population of individually sequestered or discretely identifiable cells comprises separation of a fused amplicon-containing aqueous phase from an oil phase, optionally wherein the separation comprises addition of Tris-EDTA (TE) buffer and chloroform, and performance of centrifugation;
obtaining sequence from the fused amplicons comprises use of a next-generation sequencing (NGS) method, optionally a paired-end NGS method, optionally a bead-based paired-end NGS method, optionally a sequencing method selected from the group consisting of MiSeq®, NextSeq, and HiSeq®;
obtaining sequence from the fused amplicons comprises use of a long read sequencing (LRS) method;
fused amplicon sequence data are obtained and then used to assemble a matrix of digital gene-expression measurements comprising counts of each expressed target transcript detected in each cell, optionally for further analysis;
paired transcript and exogenous polynucleotide (e.g., gRNA, RNAi agent or other exogenous polynucleotide) sequences of fused amplicons are obtained for at least 10,000 individual cells, optionally wherein paired transcript and exogenous polynucleotide sequences of fused amplicons are obtained for at least 100,000 individual cells, optionally wherein paired transcript and exogenous polynucleotide sequences of fused amplicons are obtained for about 1,000,000 or more individual cells;
the gene perturbation effects of at least 1000 different exogenous polynucleotides are assessed in the population of individually sequestered or discretely identifiable cells; and/or
the plurality of oligonucleotides further comprises a third pair of oligonucleotide primers for amplifying an exogenous polynucleotide or a second target transcript of the individually sequestered or discretely identifiable cell, optionally wherein three or more distinct nucleic acid sequences are fused.

16. The method of claim 2, wherein:
the population of droplets or emulsions comprises water-in-oil emulsions, optionally wherein the oil is an immiscible oil, optionally comprising at least one fluorosurfactant, optionally wherein the fluorosurfactant is a block copolymer consisting of one or more perfluorinated polyether (PFPE) blocks and one or more polyethylene glycol (PEG) blocks or wherein the fluorosurfactant is a triblock copolymer consisting of a PEG center block covalently bound to two PFPE blocks by amide linking groups;
the population of droplets comprises mean droplet volumes of between about 10 pL and about 1 nL per individual droplet;
the population of droplets or emulsions comprises mean droplet or emulsion volumes of between about 80 pL and about 1.2 nL;
the population of droplets or emulsions comprises mean droplet or emulsion volumes of between about 10 pL and about 80 pL, optionally wherein the population of droplets or emulsions comprises mean droplet or emulsion volumes of between about 20 pL and about 80 pL, optionally wherein the population of droplets or emulsions comprises mean droplet or emulsion volumes of between about 20 pL and about 60 pL;
the population of droplets or emulsions comprises mean droplet or emulsion sizes of between about 20 microns and about 200 microns in diameter per individual droplet or emulsion, optionally wherein the population of droplets or emulsions comprises mean droplet or emulsion sizes of between about 90 microns and about 150 microns in diameter per individual droplet or emulsion, optionally wherein the population of droplets or emulsions comprises mean droplet or emulsion sizes of between about 120 microns and about 145 microns in diameter per individual droplet or emulsion, optionally wherein the population of droplets or emulsions comprises mean droplet or emulsion sizes of about 135 microns in diameter per individual droplet or emulsion; and/or
the population of droplets or emulsions comprises mean droplet or emulsion sizes of between about 20 microns and about 90 microns in diameter per individual droplet or emulsion, optionally wherein the population of droplets or emulsions comprises mean droplet or emulsion sizes of between about 20 microns and about 70 microns in diameter per individual droplet or emulsion, optionally wherein the population of droplets or emulsions comprises mean droplet or emulsion sizes of between about 20 microns and about 50 microns in diameter per individual droplet or emulsion.

17-43. (canceled)

44. A droplet or emulsion comprising a fused amplicon comprising a target transcript amplicon joined with an exogenous polynucleotide or an exogenous polynucleotide identifier sequence amplicon, wherein the fused amplicon is formed by overlap extension amplification and wherein the exogenous polynucleotide identifier sequence is an expressed sequence that indicates the presence in the droplet or emulsion of a specific combination of exogenous polynucleotides.

45. A method for identifying within a population of individually sequestered or discretely identifiable cells one or more polynucleotide-tagged polypeptides or one or more polynucleotide tag-associated polypeptides and one or more target transcripts in an individual sequestered or discretely identifiable cell, the method comprising:
(a) preparing or providing a population of individually sequestered or discretely identifiable cells, wherein a plurality of individually sequestered or discretely identifiable cells harbors or expresses a polynucleotide-tagged polypeptide or expresses a polynucleotide tag that indicates expression of one or more tag-associated polypeptides in the cell and a plurality of the individually sequestered or discretely identifiable cells are contacted with nucleic acid amplification reagents and a plurality of oligonucleotides comprising:
(i) a first pair of oligonucleotide primers for amplifying a tag of the polynucleotide-tagged polypeptide or the polynucleotide tag that indicates the presence or expression of the one or more associated polypeptides in the individually sequestered or discretely identifiable cell; and
(ii) a second pair of oligonucleotide primers for amplifying a target transcript of the individually sequestered or discretely identifiable cell,
wherein the first pair of oligonucleotide primers possesses a primer having a 5′-terminal region of sequence that is the same or complementary to a 5′-terminal region of sequence of a primer of the second pair of oligonucleotide primers, wherein the 5′-terminal region that is the same or complementary between the first pair of oligonucleotide primers and the second pair of oligonucleotide primers is of sufficient length to allow for amplification-mediated joining of an amplicon of the first pair of oligonucleotide primers and an amplicon of the second pair of oligonucleotide primers into a fused amplicon,
wherein the individually sequestered or discretely identifiable cell is lysed to render contents of the cell accessible in a manner that maintains the sequestering or discrete identification of the lysed cell contents;
(b) performing polymerase-mediated primer extension and optionally thermal cycling upon the population of lysed cell contents under conditions suitable for generating fused amplicons comprising the amplicon of the first pair of oligonucleotide primers and the amplicon of the second pair of oligonucleotide primers by overlap extension, thereby generating fused amplicons within the individually sequestered or discretely identifiable lysed cell contents;
(c) recovering fused amplicons from the population of lysed cell contents; and
(d) obtaining sequence information from the fused amplicons using a sequencing method capable of obtaining sequences from both ends of individual fused amplicon sequences and identifying as a pair said sequences obtained from both ends of the same individual fused amplicon, thereby identifying in the population of individually sequestered or discretely identifiable cells one or more target transcripts and one or more polynucleotide-tagged polypeptides or expressed polynucleotide tag-associated polypeptides within the individually sequestered or discretely identifiable cell.

46. The method of claim 45, wherein the nucleic acid amplification reagents comprise reagents selected from the group consisting of Polymerase Chain Reaction (PCR) reagents, Recombinase Polymerase Amplification (RPA) reagents, Rolling Circle Amplification (RCA) reagents, Loop-mediated isothermal amplification (LAMP) reagents or other isothermal amplification reagents, optionally wherein the nucleic acid amplification reagents comprise PCR reagents, optionally wherein the nucleic acid amplification reagents comprise reverse transcriptase PCR (RT-PCR) reagents.

47. The method of claim 45, wherein:
the polymerase-mediated primer extension and optionally thermal cycling performed upon the population of lysed cell contents under conditions suitable for generating fused amplicons comprising the amplicon of the first pair of oligonucleotide primers and the amplicon of the second pair of oligonucleotide primers by overlap extension comprises performing one or more rounds of amplification selected from the group consisting of Polymerase Chain Reaction (PCR), Recombinase Polymerase Amplification (RPA), Rolling Circle Amplification (RCA), Loop-mediated isothermal amplification (LAMP) or other isothermal amplification, upon the population of lysed cell contents, optionally wherein PCR and thermal cycling are performed upon the population of lysed cell contents, optionally wherein reverse transcriptase PCR (RT-PCR) and thermal cycling are performed upon the population of lysed cell contents;
the polypeptides of the one or more polynucleotide-tagged polypeptides or one or more polynucleotide tag-associated polypeptides comprise one or more transcription factors;
the polypeptides of the one or more polynucleotide-tagged polypeptides or one or more polynucleotide tag-associated polypeptides comprise one or more protein variants;
the polypeptides of the one or more polynucleotide-tagged polypeptides or one or more polynucleotide tag-associated polypeptides comprise one or more protein libraries; and/or
the plurality of oligonucleotides further comprises a third pair of oligonucleotide primers for amplifying an exogenous polynucleotide or a second target transcript of the individually sequestered or discretely identifiable cell, optionally wherein three or more distinct nucleic acid sequences are fused.

48-51. (canceled)
